SEQUENCE LISTING (27 sequences)

SEQ ID NO: 1 - DNA, 1629 bp, Lactobacillus helveticus DSM 33430
atggcaaaagatattaaattctcagaaaacgcaagacgttcacttttaaagggtgttgataagttagctgatactgttaagactactattggtcctaagggtagaaacgttgttttggaacaaagttacggtaacccagacatcactaacgatggtgttactattgctaaggcaattgaattaaaagaccactacgaaaacatgggtgctaagttagttgccgaagcagctcaaaagactaacgacatcgcaggtgacggtactactactgctactgttttgactcaagctatcgttcgtgaaggtatgaagaacgttactgctggtgctaacccagttggtattcgtcgcggtattgaaaaggcaactaaggctgctgttgatgaacttcacaagatcagccacaaggttgaatcaaaggatcaaattgctaacgttgctgctgtttcatcagcttctaaagaagttggtgctttgatcgctgatgctatggaaaaggttggtcacgatggtgttattactattgaagactcacgtggtatcaacactgaactttcagtagttgaaggtatgcaatttgaccgtggttacttatcacaatacatggtaactgacaacgacaagatggaagcagaccttgataatccatacatcttgatcactgacaagaagatttcaaacattcaagacatcttgccattacttcaagaaattgttcaacaaggcaagtcattgttgatcattgctgacgatgtaactggtgaagctcttccaactcttgttttgaacaagatccgtggtactttcaacgttgttgctgttaaggcacctggctttggtgaccgtcgtaaggctcaattacaagatatcgctgctttaactgggggtactgtaatcactgaagaccttggccttgaacttaaggacaccaagattgatcaattaggtcaagctcgtcgtatcactgtaaccaaggactcaactactattgttgacggtgctggttcaaaggaagccattgacgaacgtgttgacactatcagaaagcaaattgaagactcaacttcagacttcgacaagaagaagttgcaagaacgtcttgctaaattaactggtggtgtagctgttatccacgttggtgctgctactgaaactgaacttaaagaacgtcgttacagaattgaagatgccttgaactcaactcgtgctgccgttgacgaaggttacgttgctggtggtggtacagctttagtaaatgttgaaaaggctgttcgtgaagttaagggtgaaaccactgatgaacaaactggtatcaacattgttcttagagctttgagtgcaccagttcgtcaaattgctgaaaacgctggtaaagatggctcagtaatcttagacaagcttgaacaccaagaaaacgaaattggttacaacgctgctactgacaagtgggagaacatggttgatgccggtattatcgacccaaccaaagtaactcgtaccgctttacaaaacgctgcttcaattgctgctcttcttcttactactgaagctgttgttgcagagattcctgaaccaaagccagctgctcctcaaggtggtgcaggtgctggtgctccaatgggaatgtaa

SEQ ID NO: 2 - DNA, 1965 bp, Lactobacillus helveticus DSM 33430
atggctgataattcaaagaaaaatcttgataaggtcaaaaaatatgaaaaagaggccgacaagtataatgccagtcaaattcaagtcttaggcggcctggaggctgttcgtaagcgaccaggtatgtatatcggttcgactagttctcagggattgcaccacttagtgtgggaaattattgataacagtattgatgaacgacttgctggttttgctaccaagattgaaattactgttaatgaggatggtagtgttactgttcaagatgatggtcgtggtatcccagtagatattcaaaaaaagacaggtcgtccagctttagagactgtatttactgtacttcacgccggtggtaaattcggcggtggcggatataaggtatcaggtggtcttcacggtgttggtgcatctgttgtaaatgctttgtccaccaagcttgatgtaactgtcatgcgtgacggtaaaaagtatttcatcgattttgatcacggccgcgttaaggatgaaatgaagcagattggtactgtgccacttgatcaacatgggactattgttcatttctatccagatcctgacatttttactgaaactactactttcgatgacaagatcttaaagaatagaattcgtgaacttgcatttttaaacaagggattgaaacttacttttacagataagcgtaaagatagtgccgaaaccgatgtatacaagtacgaaggtggtattaaagaatacgttgcctttttgaacaaggggcaggaagtattatttgatgaacctatctatgtcgaaagcacatataacggcattgacgtggaagttgctttgcaatatacaaatggctacaaaactactttaatgacctttgccaacaatatccatacctatgaaggcgggatgcacgaagccggcttcaagactgctttgactcgtgttgtaaatgattatgctcataaggcaaaaatcttaaaagacaaggatgataacctttctggtgaagacatccgtgaagggatgacggttgttgtttcagttaagcaccccagccctcaatttgaagggcagactaagacaaaattgggtaactcagatgctagaacagcagtagataagtcattctctgaaacctttagtcgcttcttaatggaaaacccatcagttggacgcaaaattgtggaaaagggtcaacttgccgaacgtgctagaaccgctgctaagcgtgcacgtgaagttactcgtaaaaagtctggtttggaaattgcaaacttgccaggtaaattagccgataatacaagcaatgatccaagtatttctgaattattcatcgttgagggtaactcagccggtggttcagctaagcaaggtagatctcgtttaacccaagccattttgccaatcagaggtaagattttaaacgttgaaaaagcttcaatggatcgaattttggccaaccaagaaattagatctttgtttacagctttaggtacaggatttggtgcggactttgatgtgtctaaagcacgttatcataagttgattattatgactgatgccgatgtcgatggtgctcacattagaactttgcttttgacgctgttttacaattacatgcgtccaatgattgaaaaaggatatgtatacatcgctcgtccacctctgtaccaagtccgtcagggcaaggtaattaagtatcttgatactgatgaggaattgcatgattacttaggtagtttgcagccaagtcctaagcctgttgtacagcgttacaaggggttaggtgaaatggacccagagcagttatgggaaactactatgaatccagaaaatagacgtttggatcgagtaagtcctgaatatgccaaggatgcagatgcagtctttgaactcttaatgggtaatgaagttgctccaagacgtgactttattcaaaagaatgctaaatatgttgaaaatttggatgcatag

SEQ ID NO: 3 - DNA, 1368 bp, Lactobacillus helveticus DSM 33430
ttgttcgacattaaaaaattttggcaacattttaacgatgaaatgcgagctcgttttaatgaagttgcttataatgcatggtttaaaaatactaaacctatttcttacaataaaaatacacatgaattgaaaattttagttcaaaatcctgtaacaaaaggatattgggaaaaaaatctttcttcacagttaattcaatccgcatatggatatgctggaatggaaatcctacctgtattccaaataaacgaaaataacgacagccctgagcgaatcgttactccagaacctagatatgctattcaattacgacaagaaaaaagagcacctaagcaatttactaaaaatttgaagttaaatgaaaaatatacatttgacaatttcgttcaaggtgagggaaataaattggctgccggtgctgcattagccgtagcggacagtcctggtagtttttataatccattatttatttttggtggagtcggtcttggaaaaactcacttaatgcaagcgattggtcaccaaatgctcgctgaacgaccaaatgcaaaagtagtctatatacagagtgaaactttcgttaacgattttattaattccataaaaaataaaacgcaagaccaattccgcgaaaaatacaggacttgtgacttgctgctagttgatgatattcaattttttgccaaaaaagagggtattcaggaagaatttttccatacttttgagactttatataatgatcaaaagcaaattgtaatgacgagtgatcgtctgccaactgaaattcctgatctatcagagcgtttagtttcgcgatttacatggggcttacaggttgaaattacacctccagatcttgaaaccaggattgcaattttgcgaagaaaggcagaagcagaaggcctagtcattgatgacaatactttggattatattgcttctcaagtagacactaacattagagaactcgaaggtgctcttgttaaagttcaggcatacgcaactattgaaagggcagatataaacgtcaacctagcacgagaagctctagttgatctcaaattagtacaaaaaaatcggggcttgcaaatatctaagatccaagaagtcgtcgcaaattatttccaaacttcaactgctgaattaaagggtaaaaagcgtgtacggcagattgtagtaccacggcaaatagcgatgtacttatctcgagaattaactgatgctagtttgccaaaaattggacaagaatttggcggcaaagatcataccacagtaatgcatgcctatgataaaatcgacaagcagataaaaacagatacagaaataaaaagtgcagtatttgatttaaaacaaatgattgaacactag

SEQ ID NO: 4 - DNA, 390 bp, Lactobacillus helveticus DSM 33430
atgcctgcaaagaaaacagcacgtaagcgtcgtgtgaagaagcatgttgaaagcggtgtggcacacattcactcaacgtttaataatactttagtcatgattactgacgttcaaggtaatgcagttgcttggtcttcagctggtgcattgggctttaagggtagtcgtaagtctactccatttgcagctcaaatggcagcagaagcagcagctaagagtgcaatggaccaaggtatgaaacatgtagaagtttctgtaaaaggtcctggtgctggtcgtgaatctgctattagatcacttcaagcaactggtcttgaaattactgcaattcgtgacgttacgccagttccccacaatggttccagaccaccaaaacgtcgtcgtgtttaa

SEQ ID NO: 5 - DNA, 186 bp, Lactobacillus helveticus DSM 33430
atggctaaagattacgtaacaggtaagaagaccacattcggtaacaaacgttcacacgcaatgaactcagctcgtcgtgcttggaagccaaaccttcaaaaagttcgcattcttgttgacggtaagccaaagcgcgtatgggtttcaaccaaggctttaaagtctggtaaagttactcgtgcctaa

SEQ ID NO: 6 - DNA, 1560 bp, Lactobacillus helveticus DSM 33430
atgagagtttgatcctggctcaggacgaacgctggcggcgtgcctaatacatgcaagtcgagcgagcagaaccagcagatttacttcggtaatgacgctggggacgcgagcggcggatgggtgagtaacacgtggggaacctgccccatagtctgggataccacttggaaacaggtgctaataccggataataaagcagatcgcatgatcagcttataaaaggcggcgtaagctgtcgctatgggatggccccgcggtgcattagctagttggtaaggtaacggcttaccaaggcaatgatgcatagccgagttgagagactgaacggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaattgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgccatcctaagagattaggagttcccttcggggacgctaagacaggtggtgcatggctgtcgtcagctcgtgtcgtgagatgttgggttaagtcccgcaacgagcgcaacccttattattagttgccagcattaagttgggcactctaatgagactgccggtgacaaaccggaggaaggtggggatgacgtcaagtcatcatgccccttatgacctgggctacacacgtgctacaatgggcagtacaacgagaagcgagcctgcgaaggcaagcgaatctctgaaagctgttctcagttcggactgcagtctgcaactcgactgcacgaagctggaatcgctagtaatcgcggatcagaacgccgcggtgaatacgttcccgggccttgtacacaccgcccgtcacaccatggaagtctgcaatgcccaaagccggtggcctaaccttcgggaaggagccgtctaaggcagggcagatgactggggtgaagtcgtaacaaggtagccgtaggagaacctgcggctggatcacctccttt

SEQ ID NO: 7 - DNA, 1635 bp, Lactobacillus rhamnosus DSM 33428
atggcaaaagaaattaaattctctgaggatgcacgtgccgcaatgctgcgtggcgtggatcagttagcaaacacagttaagacaacgttaggaccaaaaggccgcaatgttgttttggacaagagctatggtgcaccggaaatcaccaatgatggtgtcacgatcgcaaagtcaattgacttggaagatcactatgaaaatatgggtgctaagttagtcgcagaagttgcttccaagactaatgatattgccggtgatggtacgaccaccgcaacggttttggcacaaagcattattcgtgaaggcatgaagaacgtcactgccggtgctaacccagtcggcattcggaccgggatcgaaaaggcaaccaaggctgcagttgacgaattgcacaagatcagccacaaggttaacggcaagaaggaaattgcccaagttgcttccgtttcctcttctaatgaagaagtcggcaacctgattgctgacgcgatggaaaaagttggccatgatggtgtgattaccattgaagaaagcaaagggattgatactgaactctctgttgttgaagggatgcagtttgatcgcggctatctgagccagtacatggttaccgacaacgacaagatggaagctgatctggatgatccatatatcctgattaccgacaagaagatctccaatattcaagatattctgccactcttgcaagaaatcgtgcaacaaggcaaggcactgttgatcattgctgacgatgttgccggtgaagcactgccgaccttggttctgaacaagattcgaggcaccttcaatgttgttgccgttaaagcacctggctttggcgatcggcgcaaggctcaacttgaagatattgcgaccttgacaggtggtactgtcatcagctccgatcttggcttggatttgaaggataccaagattgaacaattaggccgtgcaggcaaggttacggttaccaaggacgacaccaccattgttgatggcgctggttccaaggatgctattgctgaacgggttaacatcatcaagaagcagattgctgatacgaccagcgacttcgaccgtgaaaaactgcaagaacgcttggctaagttagctggcggggttgctgttgtcaaggttggtgccgcaacggaaaccgaactgaaagaacgtaagtatcggatcgaagacgctttgaacgcgacccgtgctgcggttgaagaaggctatgttgctggtggtggtactgccttggtagacgtattgcctgctgttgctgctttgaaagaagacggcgatgttcagaccgggatcaacattgttcaacgtgcactggaagaaccagttcgccagattgccgaaaacgccggcaaggaaggctctgtcattgttgaaaagctgaagaaagaaaagcaaggcatcggctataacgctgcaaccggcgaatgggaagacatggctaagtcaggtattatcgacccaaccaaggttacccgctctgcattgcaaaacgctgcctcagttgcagcactgttgctgacaacagaagcagttgtggctgacaagcctgaaccaaaagataacaacgctgccgctgccggcgcaaacccagcagctggcatgggcggtatgatgtaa

SEQ ID NO: 8 - DNA, 1962 bp, Lactobacillus rhamnosus DSM 33428
gtgacggacaagaaagaatcggccgaagaaaagaaagaagaactggcagcagaatatgatgctagtcaaattcaggtgctgtcaggactcgaagcggttcgcaagcgcccaggaatgtacatcggctcaactagcagtcaaggattgcatcatttagtttgggaaatcattgataacgggattgacgaagcgttggctgggtttgcaacgcgaattgaagttgaagtcaacccggataacagtgtgaccgttaccgatgacggccgtgggattccagtcgacattcaggcaaaaaccggtcgtccggcgcttgagacggtttatacggttttgcatgccggtggtaagtttggcggtggcggctataaggtctccggcggtttgcatggtgtcggcgcttcggttgtcaatgcgctatcaacgaatctggacgtgacggttgttcgcggcggcaaacggtattacatcgactttgtgcgtggcaaggttaacacgccgatgaaagaacttggcccggcaccggagcatgagcatggcaccaaggttcattttcagccggatccggatattttcacggaaaccacaacgtttgatgataaagtcctgacaacgcggattcgtgagttggcctttttgaataaggggcttaagctgacgtttactgataagcgtgcggcgactcatgaaaagctggtgtttcattatgaaggcggcttgaaatcttatgttgatttcctgacgaagaagaaagaaaacctgctgcaggaaccgatttatgtggaaggtcaggataaaggcattacggttgaagtcgcgctgcagtatacgaatgactatcacagcacgctgttgacgtttgccaataacattcacacctacgaaggcggtacccacgaaaccgggtttaaaacggccctgacgcgggtgattaatgattatgctcggcgttccggtgcgcttaaagacagcgatgatccgctcagtggggatgacgttcgggaaggcttaaccgcagttgtttcggtcaagcatcctgatccgcagtttgaaggccagaccaagaccaaactggggaactcggatgcccgcacggtggttgatcgtaccttctccgatcacttcaacaaatttcttatggaacatccggctgacggcaagctcattattgacaaggccatgcttgccagcaaagcccgcttagctgctaaacgggcgcgtgaagtaacgcgtaagaagagcggcttggaaatctccaatctgccagggaagttagctgacaacaccagtaaagatccggaaatttccgaattgttcattgtcgaaggggattccgctggcggctcggctaagtcgggacgcagccggttgacccaagcgattttgccaattcgcggtaagattctgaatgttgaaaaagcctccatggaacgtattttggccaatgaagaaatcagaacgttattcacggcgatgggcaccggttttggtcaggactttgacattagcaaggcccgttatcacaagttgatcatcatgaccgatgccgatgtcgatggcgcccatatccggacgttgttgctgacgctgatttatcgctatatgcggccggtgcttgatgctggctacgtgtatatcgcccagccaccgttgtatcgcctgcgtcaaggtaaaatgatgcgctacatcgattccgatgaagagctacaggatattctggggcaactgccgcctagcccaaagccggatattcaacggtataaagggcttggtgaaatggacgccagccagctttgggaaaccaccatggatcctgacacgcgccgacttttacgggtatcgccaaaagatgctgaacaagcagatggcatttttgaaatgctgatgggcgatcatgttgagccgcggcggaagtttatcgaagaaaatgcggtctttgttgatccgaacaacattgacgtatga

SEQ ID NO: 9 - DNA, 1350 bp, Lactobacillus rhamnosus DSM 33428
atgcccaatttagaggagctttgggcttacctgaatgataaattccgtgaagagttgaccccagtcggttacagcacatggattcaaacagccaagccggtcaaattgacccaagataaactcgaaatcgaagtacctgcatctttgcacaaggcttactgggagaaaaatctggttaccaaagtggtggaaggcgtctatgagtttgcccaattagaagtcgatcctgtcatcatgaccaaagacgagttgcagcccgcttccacgcatcagcatcaagtaaccgatgacgatgatgaccaacaactgacctttaaggcaaaaacgcatttaaacccgaaatatacttttgaccgcttcgtgattggtaagggtaaccaaatggctcacgcggcggctttagcagtagctgaggcgcctggtacgacttacaacccgctgtttatttacggcggcgttggcttgggaaaaacgcatttgatgcaggcgatcgggaatctggtcttggaaaataatcccgctgccaacattaaatatgtcaccagcgagaattttgccaatgacttcattaattccatccaaacaaaacagcaagaacaatttcgtcaagagtaccgaaatgttgacttgctgttggttgatgatattcaattcttcggtgacaaagaagccacgcaagaagagttcttccatacatttaatacgttatacgaaaatatgaagcaaatcgtcctaaccagtgatcgcctgcctaatgagattcctaagttgcaggaacgtctggtatctcgcttcaataaaggattatccgttgacgtcaccccgccggatcttgaaacccgtatcgccatcttacgcaataaagccaatgcagaagatctcagtattccggatgacacgctttcatacattgccgggcaaattgaaagtaacgtccgtgatcttgaaggggcgctggttcgggttcaggccttttcaacgatgaagaacgaggacatcacaaccagtttagcggcagatgcactaaaagcccttaagctcgacgatcgcagcggtcaactcaccattccgcagattctcgatgcggtcgccaaatattttcaagtcaccgttcaggatctaaaaggcaaaaagcgggttaaacaaattgttattccccggcaaatcgccatgtacttagcacgagagatgaccgacaatagcttaccgaaaatcggccaggaaattggcggaaaagatcacacgactgtcattcacgcccacgaaaaaatcatggctgccatgaccaccaacgaagatttaaaagcacatgtggtcgaactacgaaatatcctgaaaaatcgtggttaa

SEQ ID NO: 10 - DNA, 390 bp, Lactobacillus rhamnosus DSM 33428
atggcaggtagaaaaactacgcggaagcgccgtgtccgcaaaaacgttgaatctggcgtggctcatatccactcaaccttcaacaacaccttagtaatgatcacggaccctcgcggcaatgcgatcgcttggtcctctgctggtgcactcgggtttaaaggtagtcgtaagtcaacaccgtttgctgcgcagatggctgctgaagctgcggcgaaggaatcaatggaacatggcatgaagtcggttgaagttgcggttaagggcccaggctctggtcgtgaagctgcaatccgttcacttcaggcaacaggtcttgaagttactgcgatccgtgatgttacaccggttccgcataacggttctcgtcctccaaagcgtcgtcgtgtttaa

SEQ ID NO: 11 - DNA, 186 bp, Lactobacillus rhamnosus DSM 33428
gtggctaaagatgttattactggtcgtcataccacgtttggcaacaagcgttctcacgctttgaattcaagccggcgccaatggaaagccaacctgcacaaggttcggatccttgttgatggtaaaccaaagcgggtatgggtttctgcacgtgcccttaaatcaggtaaagtgacccgggtttga

SEQ ID NO: 12 - DNA, 1566 bp, Lactobacillus rhamnosus DSM 33428
tatgagagtttgatcctggctcaggatgaacgctggcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctt

SEQ ID NO: 13 - DNA, 5922 bp, Lactobacillus rhamnosus DSM 33428
atgcaaacaaaaaggaaagggctatcaattctcttagccagcacagttgcattaggagcactttctgttttgccaattggtgaagttcaagctaaagcggcaatctcgcagcaaactaaaggaacaaatttagcggaagccaagactgcctcagctggacgaactgccaaagacacgaccagtgcggtgaccaatcaagccattgcgacccaattagccgcaaaaggcattgattacaacaagttaaataaagttcagcaacaggatacttatgtcgatgttattgtgcagatgagcgcggcaccggcttccgaaaatggcacgctcaaacccgattactccagtactgccgaaattcagcaggcaacaaatcaggtcatcgcggctcaagccagtgttaaatcagccgttgaacaagtgacgcatcagacagctggagaaagctatggttacgttgttaatggctttacaaccaaagtcaaagtcgctgatattccgcggttgaaacaaatccccggcgttaaaaccgttactttagccaaggtttattacccaaccgatgctaaggccaattcaatggcaaatgtgcaggcggtttggtctaattacaagtataaaggggaaggcacggttgtctcggtcattgatactgggattgaccccaaccataaggacatgcggcttagtgatactaaaaaggccaaattgacccgaacagatgttgaaaaattcaccagtactgctaaacacgggcgttatttcacggctaaggttccgtatggctttaattacgcagataacaatgacacgattaccgacgacacggttgatgaacagcatgggatgcatgtttccgggatcatcggggctaacggtacaggtgatgatccaaccaagtcagttgtcggtgtagcgccagaatcacaattattagcgatgaaggtgtttaccaattcagatacatcggcgacgactggttcgtcaacgcttgtttctgcgattgaagattcggccaaactgggcgcggatgttttgaatatgtcccttgggtctgtttccggcaatcaaacactggaagatcctgaaattgcagccgttcaaaacgccaatgaatccgggaccgcagcggttatttcagcaggaaattcgggtacatccgggtcaggtacagaaggggtcaacaaagattattatggcctgcaggataatgagacggttggcacaccggggacatcacgcggggcaacgactgtcgcatcggccgaaaatacagatgtcatcaaccaagccgttacgattactgacggcagcgggttaaaactcgggcctgaaaccgtacagctttcaagcaatgactttgttgacagttttgatcagaagaaattctacgttgtcaaagacgcaagcggtaagttaagtacaggtgatgctggcgactatacggcggatgccaaagggaaaattgcaattgtcaaacgtggcagtctgactttcactgacaagcagaaatatgccgaggccgctggtgcagcaggcttaatcattgttaataatgatggcacatccacacctttgacttcgatttctctaacggctacttttcctacttttggtctttccaatacgactggccaaaaactggttgactgggtaactgcgcatccaaatgacagtctgggcgtaaaaattgccttggcgttgttgccaaatcaaaattataaagcagatcggatgtcaagtttcacttcatatggccctgtatctgatctttcctttaagcctgatattacagcgccgggtggtaatatttggtcaacgcaaaacaacaatggttatacgaatatgtcggggacctcaatggcatctccatttatcgccggctcccaggcacttctaaaacaagcgctaaataataaagacaatgagttctatgccgactataagcaacttaaaggcacagcattaaccgatttcctcaaaacagttgaaatgaatactgcaaaaccgattaatgatattaactataataatgtcatcgtctctccacgccgacagggagcagggttggtggatgtcaaagccgcgattgatgctttggagaagaatccatcaacggttgtttcggaaaacggctatcctgctgttgaattgaaagatttcacgagcactaccaaaacgtttaagttgacgtttaccaaccgcaccaagcatcagctgacgtatcaaatgactagcaatgaagacaccaatgcggtttatacttcggctaccgatccgaattcaggcgttctctatgacaagaaaattactgatgcggcagtcaagccggataccgatattgtggtaccggctggaaaatccgtgaaggtaccgttcacgctttcactgcctaagacttttgatcagcaacagtttgttgaaggattcttgaacttcaagggaaatgatggctcgaatctcaacttgccatatatgggcttctttggtgattggaatgacgggaagatcgtggatagcctgaatggcgtgacttacgatccaactggtggcaattatgggactgttccggtgctcaccaataagaaaaccggcagtcagtattacggtggcttagtcacggatgcaagtggcaagcaaacggttgatgataaagcagtggccttttcgagcgataagaacgcgctttataatgacatcagcatgcaatactacctgttacgcaatattagcgatgtgcaagtcgatgtgctggataatcacggtaacaaagtaacaacgctcagcagtacgactaaccagactaagacttattatgattcaactggtcgcaactatacttactatcgcgcaccggcttgggatggtacgtattatgatcaacgagatggcaatatcaaaacagccgctgatggcaactacacgtaccgtatttcgggtgttcctgaaggcggcgacaagcggcagacatacgatgtagcattcacgctggactctaaggcgccaacggttcgtcatgtcgctttaacgtctagacaggaaaaaggcaaaaccacttactatctcaccgctgaagcaaaagacgatcgaagcggtttggatgccactaagagtgcaaaaacatcggttaatcaagtcaccaatcttgatggcacctttacgacgacagggacaactgctgatggctacacaaaactcgaaacgccactgacggataagcaggcacaagccttgggtcaaggcgataataatgttgaactttatttgaccgataacgcctcaaacgccacggatcaatcagcttccgcacaaaagcccggatcaactgcttatgatctgattattaatggtggtggtttaccagataaaatcacgagtcaaacggctaactatcaagcagggaagcaaggtggcacttacacctttaccgggacgtatccggctgctgtttatggtacctataccgatgcgcaaggaaagcagcatgatttgacgaccacttatgacgctgatgccaatagtttcaaagccactatggcacttgacgccagcgactatgcgacgaaggttgatctttatactgaccaagcacatacccagttagttaaacattttgatacgaatgtccggttggcggcacctacttttagtgacttgcgggttaatgataatcaagatcaaacgtccgaaagtaccgttaaagtaaccggaacagctagtgccgacactgagaaggtaacggttactaacggtggcacgacaacaccggttactttggatgccaaacatcactttagtggcgatgtacccgttaactatggggataataccattacggtgacggcagaagatgaagatggcaataccgttaccaagcagcagaaggtaaactcaacttatgatgccgatgtcctaaaaaatgcggtgaccttcgaccaaggcgtcactttcggggcaaatcaactgaaagttaaagatgctaagtactatgaccctaagaccgggattgcaacgattaccggtaaagtcaagcatccgacgacgactttgcaagtagatggtaaacaagtaccgattaatgatgatctaacctttagttttaagttgaatctggggacagccggtcagaaaccgtttggcgttgtaattggtgataccacgcaagataaaacggttcaagacagcttgacctttattttagatgctattgccccgacactgtcactcgatagctcgacggacaaacctgtctatacgaatgatccgaatttccgtatcaccggaacagcgacagataacgttaattatttggaactggcaattaacggtagtcaggttgccagtcaatatgaggacattaacttaaacagcgggcagccgggtcatatggcaattgatcagacggtgaaactattagagggcaagaatgtcttaaccgttgcggcaacagatagcggcagtaacgtcacaactaaaaagattacggtttattatgagcctaagaagacactcgcagcgccaacaattacgccaaatacgactgatccagctaaagaagtgacgttgacagccaaggcagcagcaggtgagaccgttcaatacagtactgatggcggcaagacttaccaagatttaccgacaacgggactgagtgtatcggcaaacgcggactttaaatttaaggctgttgatctatacggtaatgaatcgccggcagtagattacgcggttaagaatattaagacggatgatccggcgcagttacaaaaggccacaacaacgctgcaggatttgcttactcaagcaaaggcaaaagcagcgagcgctcagtatactgacgccacaactgatgctctgaataccgctatcggatcggcgcagactgctttagctaaagctgatgccacaattgaaacactcacaacggcaacaacgcagctgaccaccgcagtgaatcagttggttgataagctgccagcagaccaacaagctgcactactaaacaagatacaatctgccaaagaggcgtttggaactgatttaggtggccaaaccgatccaagcaccggtaaaacgctaaatgccgaactggacgctgtcgctgcgcaaacgacagcaggaactagtaccgccgatcaaattgaaaccaacttcaataaggtattagatgctgcattgaatcaattggcaaaaacaattaaagcggcaacgcctgtcaaagttggtaatgccaaagataccactaccggtaaaacctggtacggtgatgttgatgctgtcattgctgcaggtacagcagctaaagcggatactgaaaagattgcacaattgcaaggtttgtttggtttgaagactaaaattgccgcagcggttgaggcagctgccaagacaccacagcaacctggaggcggatccggaagtggttctgacactggcaaagggtctggcaccggaagcggctctgaagcaggtaaaggttcaggcaccggaagtggttccgaagcaggtaaaggatctggcaacggaagtggatcagaagcaggtaaaggatcaggcaccggaagcggttctgaagcaggtaaaggatcaggcactggaagtggttccgaagcaggtaaaggatctgacaccggaagcggttcagatgccggtaaaggctctggcactgacaagggaaatcaaccaaaagacacaccatcaaccaatcctaagggcggcgacgacaagaaacagacgcaagaaacgccggcacaaccaacagggaccgaaaacgccaatagtaatggtgcaaacagtcaagcgtccactaaagatacgctgccaagtacaaatgaatcgccacggcccgcacttgctttcttaggagctttagtcatgggtgggctaggtcttcttggcatcaagcgtaaacgcaagcaaagctaa

SEQ ID NO: 14 - DNA, 456 bp, Lactobacillus rhamnosus DSM 33428
atggtcaaaccgattacattagatgaacaactgtgttttgcgatttacaaggcacaaaaacaatataaccatttttacgcgcaggcattggcaccgtttaagctgacttatccgcaatatatcaccctgttgtctttatatgaacacggtacgatgtcggttaaacaagtcggacaaacgcttgaactggacagtggtaccttaacgccattgatgaaacggctggagaaagacggctggatcaatcgcaaacgctcaactgaagacgagcgtcgcgttgatgtgagcctgacgaaaaaggctttggatgcccgcgatcagatttttgaacatgttggctcgtgcatgaaactgatgaatctgccgcagtctgaatatgatcatattaaatccgaagtagcgcgggtggacgaacatttgagtgctatcgctgataatgcttatcccgaagcataa

SEQ ID NO: 15 - DNA, 879 bp, Lactobacillus rhamnosus DSM 33428
atggaacaagttcagccaaacaaagcctttttctggggtattctgcttaatttgatttacattattgccgaggttgcgtttggaatcgggattggctcggttgcgttggtcgccgatgccattcataatatgagcgacgtcctaggcttgggcttagcgtggtttgcggagtggctttccaatcggcacccgactgtccagcgtacatacgggtataagagttcgtcgattttagcggcgttagcaaatgcggcatttttactggtggcgatgggtgcgattattgtcgaggcgattacccgattgtcacaaaatgccccggttgaaggcggctggatgatggtcgtcgctggtgccgggattttggtcaatggcgcaactgcaattctttttcatagcggctcgcaacatgatttgaacattcgtggtgcgtttatgcacatggctgccgatgctggcgtgtcgctgggagttgttttagccgggggattaatcctgctgactggctgggaatggcttgatccggtcatgtccattttagttgcaattgtggttttgatcgggacttgggatttattgcgggatgccgttaatttagcaatggccgcggtgcctaaggaaatcgatccagtggcggttaaaacattggttaacgcctatccaaccgttgcatcttgccatgacctgcacatctgggcgctgagtacaactgatgtcgcgttgacggtacatgtggtgcgaacaactgctgaaggcaatgacgccttcttggacgcactgagtttatcgttgcgagaaagctttgacattgcccacaccacgattcaagttgaatatgggccgtatttaccggataacaaaatggagaatccttactga

SEQ ID NO: 16 - DNA, 1623 bp, Lactobacillus mucosae DSM 33450
atggcaaaagaaattaagttttctgaagatgcacgcaaggcaatgctgcgtggggtagacaagttggccaacacggttaagacgacgatcggtcctaagggtcgcaacgttgttttggaacaaagctatggcgcgcctacgattactaacgatggggttacgatcgccaagaacattgaattggaagaccactttgagaacatgggtgccaagcttgtttctgaagttgcttccaagaccaatgacaacgccggtgacggtacgacgacggcaaccgttttgacgcaagccatcgttaacgccgggatgaagaacgttgctgccggtgctaacccggttggcattcgtcgcggtatcgacaaggctaccaaggctgccgttgaagaactgcacaagatgagtcatgaagtgcaaagcaaggatgacattgcgcaaattgcctccatctctgctgctaaccctgaagttggtgaactgattgccgatgccatggaaaaggttggccacgatggcgtaatcacgattgaagacagccgtggcgttgacacgacggttgacgtggttgaagggatgagctttgaccgcggctacatgtctcaatacatggtaaccgacacggacaagatggaatctgatctggacaatccatacattctgatcactgacaagaagatcggcaatattcaagacattctgccattgctgcaggccgttgttcaagaaggccgttcgcttttgatcattgctgacgacattaccggtgaagctctgccaaccttggtattgaacaagattcgcgggacgttcaacgtttgcgcggttaaggcgccaggctttggcgatcgtcgtaaggctcaattgcaggatatcgccattttgactggtggtaccgtaatctctgatgatctgggcatgagcttaaaggatgctacgattgctcaacttggttctgctaacaaggttaccgtaactaaggacaacacgacgattgttgatggcaacggtgctaaggacgcaattgctgaacgagttgaacagatcaagcaggcaattgccaacacgacttctgaattcgacaaagaaaagctgcaggaacgtctggccaagctgtctggcggggttgccgtaatcaaggtcggtgctgctactgaaacggaactcaaggaaaagaagtaccgcgttgaagatgctctgaacgctacgcgggctgccgttcaagaaggtttcgtacctggtggtggtactgctttgatcaacgtaattcctgccttggaaaacgttgaaactgaaagcgacgacgaagcaactggtgtcaacatcgttaagaaggcattggaggccccagttcgtcaaatcgctgaaaacgctggcgttgaaggctcaattatcgttaaccgcttgaagagtgaaaagccaggcatcggttacaacgctgccgttgacaaatttgaagacatggttgcggccggtatcgttgaccctaccaaggttacgcgttcagcactgcaaaacgctgcttcggtatcagcaatgcttttgaccactgaagccgttgttgctgataagccagaaccaaagaatgatcagccagctgctcctcaaggcggcgccggtatgatgtaa

SEQ ID NO: 17 - DNA, 1953 bp, Lactobacillus mucosae DSM 33450
gtgagcgataaagaacttgaagcagaaaagaaagcggaactggccagtgagtacgatgccagccagattcaagtcttaaaagggctggaagcagtccggaagcgccctgggatgtacattggctccaccagtgctcaagggctgcatcacctggtctgggaaatcgtcgacaatggtattgacgaagccttggccggattttgtgatgagatcaatgtcgttgttgaaccagacaacagcattacggtaaccgacaacggacgggggatcccgatcgatattcaaaaagaaaccggcaaaccggcactggaaacggtctttaccgttctacatgccggtggtaagttcggcagcggcggctacaaggtttctggtggtctgcacggggttggggcctcagtcgtcaacgcgctgtcaaccgagctggatgtctacgtaacgcggcaaggcaaggaatactatatggactttgaccatggccacgttaagacgccgatgaaagtcgttaaagagggcgtctcggaattaaagcacggcacccgggttcactttaagccagatccagaaatcttccgtgaaacgacggtctacaacatcaagacgctgacggatcgaattcgtgaattgtcttttttgaacaagggcctaaagatcacgattgaggacaagcgagaagaaaagccaacccggcaagagttccattatgaaggcgggattcggcactacgttgaattcttaaatgaaggccaagaagtcctgtttgatccgccaatctatgtggaaggcgaggacaagggcatcacggttgaggtttccctgcagtatacgacgggataccgcagcacgctgatgacttttaccaacaacattcacacttatgaaggcggaacgcacgaaaccggctttaaaaccgctttgacgcgcgttatcaatgactatggccgaaagagcggtcagctgaaggggaatacgacgctttccggtgatgatgtgcgtgaaggattgacggcagtcgtttcaatcaagcaccctgatccacagtttgaaggacagaccaagaccaagctgggcaactcggatgcacggacggcaaccgaccacatcttcagcgagatgtttgcgcgctttttgatggaaaatccggacacagcgcacaagatcgttgaaaagggaatgctggcttccaaagcgcgggtagccgccaaacgggctcgtgaagttacgcgtaagaagagcggcttggaaatttcaagattacccgggaaattagccgacaacaccagcaaggatccagaaatttcagaattgttcatcgtcgagggtgactcggccggtggtagtgccaagcagggccgttcccgcttgacgcaggcgattctgccaattcgtggtaagatcctcaacgttgaaaaagctgggatggaccgcattctggccaatgatgagattcgttcgctgtttactgctctagggactggttttggcggtgattttgacattaccaaggccaactaccacaagctgatcatcatgaccgatgccgatgtggacggtgcgcacattcggacgctgctgctgacgcttttctatcgctatatgaagccaatgatcaccaatggctatgtctatattgcacagccgccactgtatcaagttcggcagggcaaattcgtcaagtacgtcgaatcagacgaagagctgcaaaacgtcctgaacacgctgcagccaagccctaagccggtaattcaacgctacaaagggctgggtgaaatggatgccgaacagctctgggaaacgacgatggatccagaaaaacgccatctgctgcgagttgacctggatgacgctgctgaagcggaaaagatcttcccaatgctgatgggaaccaaagtcggtccgcggcgcgagtttattgaagaaaatgccaagtttgtcgagaacttggacctataa

SEQ ID NO: 18 - DNA, 1311 bp, Lactobacillus mucosae DSM 33450
ttgattgagctaaactcactctgggaatcggttcaaaaagagtttcgcaagaccactggcagcgtaacctataacaacctaattgccccagccaaagcaatttctctcgaaaacaatcagctgaccattgagctgccaaccaaatctcaccgggacttttggcaaaacgagatgaccgctaagctcaaggacctgctgttcaatgaaaccctggttcatattgagccacgctacacgcttgagagagagctcatgactccatcttcgccagccggtggaatcatgaccaaacagacacccttaaatccagaatatacatttgaagctttcgttgaaggccgcagcaatcagatggcctatgcttctgcctatgctgcctctgaacagcctggcggactttataatccactcttgatctatggtggcgtcggccttggcaagacgcatttgatgcaggccattgccaacaacatgctacggcataatcccaacgccaagatcaaatacgttaccagcgaaaccttcaccaatgactacatcaattcgatcaaaaacaacaaccaagagcagttccggcgcgagtatcgtgacctggacgctttgttggtcgatgatgtccagttcttttccaataaggaaggcacccagctggaatttttcaatactttcaattcgctgcatgacaacaacaagcagatcgtcttgaccgctgaccggaatcccaagcagatccctgatttgactgatcggctggtctcacgctttgtctggggagttccagtcgagattacctcgccagatctagaaacacggaccgccattttgcgcagcaaggccgaagaagagcagatcaacgttcccaatgacgttttaagctttattgccagcaaggtcgacaccaacgtccgcgagttggaaagcgcgctgctgagagttcgcgtagccgcccagcttcatcacgcgcccatcacgattgaactggcaaatgaaacgctcaagggcatggaagctgccgatgaggcggtaatctcgattgacttgattcaaaaaaaggttgcggcttattataatatccgtcagtctgatatcgtggggaaaaaacgcgtcaagcagatcgtctttccgcgccagattgccatgtatcttgcccgagagctgaccgaaagctcactgcctaagattggcaaggaatttggcggcaaggatcataccacggttctccatgccatcgataagattgaaaaacagctggccgaggatgataatttacaagacgacatccgcaaccttagaatggagctgcagccttga

SEQ ID NO: 19 - DNA, 390 bp, Lactobacillus mucosae DSM 33450
atggcaaccaaaaaaggttcacgcaagcgtcgtgcaaaaaagaatgttgaaactggtgttgcacacattcactcaactttcaacaacacgttgatcatgatcactgatgtgcaaggcaatgccgttgcttggtcctcagccggtgtgctgggcttcaagggctctcgtaagtctactccatttgctgctcaaatggcttctgaagctgctgccaagcaagcaatggaacacggtatgaagacggttgaagttgaagttaagggccctggttcgggtcgtgaatcagctatccgtgctctgcaagcaactggtcttgaagttactgccatccgggacgtaactccagtgccacacaatggttctcgtcctccaaagcgtcgtcgtgtctaa

SEQ ID NO: 20 - DNA, 186 bp, Lactobacillus mucosae DSM 33450
atggctaaggatttcatgaccggtaagcggacgcgcttcggcaacaagcgttcacacgcccttaactcaagtcgtcgtacctggaagccaaacctgcaaaaggttaccattttggtaaatggcaagccaaagaaggtttacgtttcagcacgtaccctgaagtcgggtaaagtgactcgggtttag

SEQ ID NO: 21 - DNA, 1574 bp, Lactobacillus mucosae DSM 33450
tatgagagtttgatcctggctcaggatgaacgccggcggtgtgcctaatacatgcaagtcgaacgcgttggcccaactgattgaacgtgcttgcacggacttgacgttggtttaccagcgagtggcggacgggtgagtaacacgtaggtaacctgccccaaagcgggggataacatttggaaacagatgctaataccgcataacaatttgaatcgcatgattcaaatttaaaagatggtttcggctatcactttgggatggacctgcggcgcattagcttgttggtagggtaacggcctaccaaggctgtgatgcgtagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttagagaagaacgtgcgtgagagcaactgttcacgcagtgacggtatctaaccagaaagtcacggctaactacgtgccagcagccgcggtaatacgtaggtggcaagcgttatccggatttattgggcgtaaagcgagcgcaggcggtttgataagtctgatgtgaaagcctttggcttaaccaaagaagtgcatcggaaactgtcagacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatggacggtacaacgagtcgcgaactcgcgagggcaagctaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgcaacacccaaagtcggtggggtaacccttcggggagctagccgcctaaggtggggcagatgattagggtgaagtcgtaacaaggtagccgtaggagaacctgcggctggatcacctccttt

SEQ ID NO: 22 - DNA, 390 bp, Lactobacillus johnsonii DSM 33448
atggctgcaaagaaaacagcacgtaaacgccgtgtaaagaagcatgttgaaagcggcgttgctcatattcattctacgtttaacaataccttggtcatgattactgatgtacaaggtaacgcagtcgcatggtcttccgctggtgctttaggttttaagggtagtcgtaagtctacaccatttgctgctcaaatggccgcagaagcagctgcaaagtctgcaatggatcaaggcatgaagcatgttgaagtttcagttaaaggcccaggtgcaggtcgtgaagctgctattagagcacttcaagctgcaggtcttgaaatcactgctattcgcgacgttactccagtgccgcacaacggttccagaccaccaaaacgtcgtcgtgtctag

SEQ ID NO: 23 - DNA, 348 bp, Lactobacillus johnsonii DSM 33448
atggcacgtattgctggtgttgacttacccagaaataaaagagttgtagtcgcattaacttatatctatggtattggtgaaccaactgcaaaaaagatttgtaaagatgctggtatttctgaagacattcgtactaatgacttgactccagaagatcaagaaaaattacgtagcgaagtagacaagtaccgtgttgaaggtgatcttcgtcgtgaagttagccttaacatcaagcgtttagttgaaattggctcataccgtggtattcgtcaccgtcgtggcttaccagttcgtggtcaaaataccaagaataatgctcgtactcgtaagggttcaaagagaaaataa

SEQ ID NO: 24 - DNA, 837 bp, Lactobacillus johnsonii DSM 33448
ttggcaattatcaaatataagccaactacaaacggcagacgtaatatgacttcttccgactttgctgaaattaccaagaaaaagcctgaaaagactttacttgaatctcaaagtcatactgctggtcgtaactcatacggtcatattactgttagacaccgtggtggtggtcacaaacaaaaataccgtatcattgacttcaagcgtaacaaggatgatgtaaaagcagttgttaaggctatcgaatacgatccaaacagaactgctaatattgctcttcttcactacactgatggtattaaagcttacattttagcacctaagggtctaaaagttggtgctgttgttgaatctggtccagatgctgatattaagccaggtaatgcattaccattatctgctattccagctggtactgaaattcacaatattgaattaaagcctggtaaaggtggacaattagtaagaagtgctggtactgttgcacaagttcttggtaatgatggtaagtacactttagttagacttcaaagtggtgaagttcgtaagattttatcaacttgccgtgctactatcggttctgttggtaatgaacaacactcattaattcaattaggtaaagctggtcgtagtcgttggttgggtaaacgtccacaatctcgtggttccgtaatgaaccctaacgatcacccacatggtggtggtgaaggtaaggctccagttggtcgtccacaacctatgactccatggggtaagaagtctcgtggtattaagactagaaactctaaggctagaagtgaaaaacttattattcgtcaccgtaaaggtaacaaataa

SEQ ID NO: 25 - DNA, 882 bp, Lactobacillus johnsonii DSM 33448
atgtacaatcatattaaagaaattgttgcagacgctgaaaaaacagaaaaacctatttcagaattaataattgaacaagaatgcaaattatcaggtctatctagagaaaaagtatggaataaaatgaaatataatcttgaaaccatgcgtgctgcggttagtaagggaagtactggtaaaggagtattttctcatactggcttaacaggtggtgaagcgattaaaataaagaattatcgtgaaaaaggacatactctttctggtgattttatgatggaagctgtgcaaaatgccgtagcgacaaatgaagtaaatgctgcgatgggcgtcatttgtgcaactccaacagctggctcttctggcacactgccaggcattttatttatgcttgaaaaaaagctagacttaaatgaagagcaaatgattcgctttctctttactgcaggaggaatgggattagttattggaaataatgcggaaattgctggtgcaaccggtggctgccaagcagaagtaggttcagcttccgcaatgggtgcagcagcagctgtagaaatagccggtggcacgcccgcgcaaagctctcaagctttagcgattgcgatgtctaatttattgggactagtatgtgatccaatagcaggactagtagaagtaccctgcgtaaaaagaaatgctattggagcaggcaacgcactaattgcggctgatatggcactagctggatgtaccagtgttatcccggctgatgagtgtattgatgctatgaaaaaagttggtcatcaaatgccagtatcattaagagaaactgggattggtggattagcaggtactccaacaggacaggctattaaggctagaatttttggaaaagatgtttag

SEQ ID NO: 26 - DNA, 186 bp, Lactobacillus johnsonii DSM 33448
atggcaaaagacattattactggccgcaagacggtctttggtaacaagcgttcaaaggctttgaactccgtgcggcgttcatggaagccaaaccttcaaaaagttcgcatccttgttgacggtaagccaaagcgtgtttgggtatccgctcgcgctttgaaatccggtaaggttaaacgtgtctaa

SEQ ID NO: 27 - DNA, 1417 bp, Lactobacillus johnsonii DSM 33448
ccaagagactgggataacacctggaaacagatgctaataccggataacaacactagacgcatgtctagagtttgaaagatggttctgctatcactcttggatggacctgcggtgcattagctagttggtaaggtaatggcttaccaaggcgatgatgcatagccgagttgagagactgatcggccacattgggactgagacacggcccaaactcctacgggaggcagcagtagggaatcttccacaatggacgaaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctcttaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaagtcgtaacaaggtagccgtaggagaacctgcggctggatcacctccttt

(19) United States
(12) Patent Application Publication
(10) Pub. No.: US 2024/0156882 A1
(43) Pub. Date: May 16, 2024

(54) PREPARATIONS COMPRISING PROBIOTIC STRAINS AND ANTHOCYANINS

(71) Applicant: EVONIK OPERATIONS GMBH, Essen (DE)

(72) Inventors: Heike TOM DIECK, Friedrichsdorf (DE); Michael SCHILLING, Berlin (DE); Michael SCHWARM, Alzenau (DE); Bodo SPECKMANN, Kahl (DE)

(73) Assignee: EVONIK OPERATIONS GMBH, Essen (DE)

(21) Appl. No.: 18/279,995

(22) PCT Filed: Feb. 28, 2022
(86) PCT No.: PCT/EP2022/054994

§ 371 Date: Sep. 1, 2023

(30) Foreign Application Priority Data
Mar. 4, 2021 (EP) 21160596.9

(51) Int. Cl. A61K 35/747; A23L 33/135; A61K 9/48; A61K 31/7048

(52) U.S. Cl. CPC A61K 35/747; A23L 33/135; A61K 9/4891; A61K 31/7048

(57) ABSTRACT

The current invention concerns preparations comprising at least one probiotic strain belonging to the species Lactobacillus helveticus, Lactobacillus rhamnosus (Lacticaseibacillus rhamnosus), Lactobacillus mucosae (Limosilactobacillus mucosae), and Lactobacillus johnsonii and anthocyanins.

The current invention concerns preparations comprising at least one probiotic strain belonging to the species Lactobacillus helveticus, Lactobacillus rhamnosus (Lacticaseibacillus rhamnosus), Lactobacillus mucosae (Limosilactobacillus mucosae), and Lactobacillus johnsonii and anthocyanins.

Nutrition is an important contributor to the health of humans and animals. The gastrointestinal microbiota acts as a relevant mediator of nutrient—as well as active pharmaceutical ingredient-triggered health effects and has therefore emerged as a target of interventions to improve health. Microbiota-targeted strategies include the application of prebiotics, probiotics and synbiotics to modulate the microbiota's composition and activity. A host-centered definition of prebiotic describes it as “a substrate that is selectively utilized by host microorganisms conferring a health benefit” (Consensus definition by the International Scientific Association for Probiotics and Prebiotics (ISAPP)) [1], thus referring not only to certain carbohydrates but also to e.g. amino acids, peptides, and polyphenols as prebiotics. Probiotics are defined as: “live microorganisms that, when administered in adequate amounts, confer a health benefit on the host” (ISAPP definition) [2]. The most commonly investigated and commercially available probiotics are mainly microorganisms from species of genera Lactobacillus and Bifidobacterium. In addition, several others such as Propionibacterium, Streptococcus, Bacillus, Enterococcus, Escherichia coli, and yeasts are also used. Synbiotics refer to food ingredients or supplements combining probiotics and prebiotics in a form of synergism, hence synbiotic [3]. In the context of this invention, we understand the term “synbiotics” as combinations of probiotics with any chemically defined substance(/s), e.g. anthocyans.

Anthocyans are polyphenolic compounds categorized as anthocyanidines (e.g. cyanidin, delphinidin) and the corresponding O-linked glycosides, anthocyanines. Anthocyans occur in many fruits, especially in intensely colored berries like bilberries, blackcurrant, and strawberries.

Anthocyans reportedly reduce levels of circulating pro-inflammatory cytokines, improve endothelial functions as well as cholesterol levels. Consumption of anthocyans from berry fruits has consequently been linked to the prevention/amelioration of cardiovascular diseases, type 2 diabetes, traits of the metabolic syndrome, and decline of cognitive functions [4-6]. These effects were substantiated by meta-analysis of 41 human trials wherein interventions with berry preparations were performed [7]. Interestingly, a high inter-individual variability in the physiological response to such interventions has been found [8]. Factors which affect the pharmacokinetics of polyphenols, e.g. dietary habits, lifestyle factors, host genome, as well as gut microbiota composition & activity, contribute to this variability. The latter aspect appears crucial, as the majority of ingested polyphenols is not absorbed in its intact form but reaches the large intestine and is converted to absorbable metabolites by gut microbes [9-11]. Kinetic studies performed with e.g. radiolabeled cyanidin-3-glucoside and blueberries revealed that anthocyanins and other polyphenols [9, 12, 13] are extensively metabolized by the host as well as by gut microbes—albeit for most of the metabolites it is largely unknown whether they derive solely from host or microbial metabolization or from a combination of both.

The prior art discloses O-deglycosylation of anthocyanidins to the respective anthocyanins by gut microbiota and by specific bacterial strains of e.g. the genus Lactobacillus [14]. Recently, the taxonomic classification of several species of this genus has been updated, according to Zheng J, Wittouck S, Salvetti E, Cmap Franz H M B, Harris P, Mattarelli P W, O'Toole B, Pot P, Vandamme J, Walter K, Watanabe S, Wuyts G E, Felis M G, Ganzle A and Lebeer S, 2020. A taxonomic note on the genus lactobacillus: description of 23 novel genera, emended description of the genus lactobacillus Beijerinck 1901, and Union of Lactobacillaceae and Leuconostocaceae. International Journal of Systematic and Evolutionary Microbiology. https://doi.org/10.1099/ijsem.0.004107. Of particular relevance in the context of this invention are the following species:

| “Old” denomination | Updated denomination (since 2020) |
|---|---|
| Lactobacillus helveticus | Lactobacillus helveticus |
| Lactobacillus johnsonii | Lactobacillus johnsonii |
| Lactobacillus mucosae | Limosilactobacillus mucosae |
| Lactobacillus rhamnosus | Lacticaseibacillus rhamnosus |

Hanske et al. disclose the formation of protocatechuic acid from C3G by a complex microbiota [11], whereas the taxa that are responsible for this effect have not been identified.

CN107058409A discloses Lactobacillus rhamnosus (Lacticaseibacillus rhamnosus) YYJP-2 and Lactobacillus casei KFL2YYJP-2 as producers of protocatechuic acid from anthocyanins derived from extracts of fresh purple sweet potato, violet cabbage, mangosteen shell, black wolfberry, or mulberry.

Cheng et al. reported net production of ferulic acid from mulberry anthocyanins by strains of Lactobacillus plantarum, Lactobacillus acidophilus, and Lactobacillus delbrueckii of up to 10 mg/l [15].

Health effects of some metabolites, such as ferulic acid and protocatechuic acid, are inferred by mechanistic and animal studies [16, 17]. Translation of these preclinical findings towards improving human health has however shown to be challenging. Direct delivery of AC metabolites by intravenous injection is not feasible for humans, particularly not in the context of preventive approaches. We reason that the directed conversion of AC to AC metabolites is a crucial step which is decisive for delivering successful outcomes from any interventions aiming to prevent, cure, or treat health conditions with AC. We conceive that the health benefits associated with AC intake can be targeted and boosted by the application of compositions that are part of this invention and are characterized by 1. The use of a source of AC with specified content and composition of individual AC, 2. Combination of such sources with selected probiotic strains that specifically produce AC metabolites of interest, 3. These metabolites being rapidly and preferably concomitantly produced by the selected microbial strains, 4. Application of colon-targeted release formulations as part of these compositions to facilitate the metabolization of AC by the probiotic strains of the compositions, in favor over their metabolization by mammalian or other microbial enzymes or their spontaneous degradation. This approach will also overcome the observed variability of AC effectiveness that results from the interindividual diversity of microbiota compositions.

The objective of this invention is to provide a technology that promotes the beneficial metabolization of AC by potent gut microbes inside an organism to provide a benefit for humans and animals by preventing or ameliorating the above-mentioned conditions. In search for potent probiotic strains in the context of this invention, we focused on producers of ferulic acid (FA), protocatechuic acid (PCA) and phloroglucinaldehyde (PGA).

| Anthocyan metabolite | Inventory name | CAS-No. |
|---|---|---|
| Ferulic acid | 4-Hydroxy-3-methoxycinnamic acid | 1135-24-6 |
| Protocatechuic acid | 3,4-Dihydroxybenzoic acid | 99-50-3 |
| Phloroglucinaldehyde | 2,4,6-trihydroxybenzaldehyde | 487-70-7 |

Therefore, it was an object of the present invention to provide a composition which supports enhanced production of ferulic acid (FA), protocatechuic acid (PCA) and phloroglucinaldehyde (PGA).

We identified strains of the genera Lactobacillus helveticus, Lactobacillus rhamnosus (Lacticaseibacillus rhamnosus), Lactobacillus mucosae (Limosilactobacillus mucosae) (Limosilactobacillus mucosae (Limosilactobacillus mucosae), and Lactobacillus johnsonii with particularly high capacity to produce FA, PCA, PGA, and combinations thereof. The production of certain combinations of metabolites is of particular interest as these metabolites may act synergistically. The strains have been identified by screening of naturally occurring isolates. Those strains have been deposited at the Leibniz-Institut DSMZ Deutsche Sammlung von Mikroorganismen and Zellkulturen GmbH, Inhoffenstr. 7 B, 38124 Braunschweig, Germany under the provisions of the Budapest Treaty on the International Recognition of the Deposit of Microorganisms for the Purpose of Patent Procedure under the Accession Numbers Lactobacillus rhamnosus (Lacticaseibacillus rhamnosus) DSM 33428 (Lakto 018), Lactobacillus helveticus DSM 33430 (Lakto 019), Lactobacillus johnsonii DSM 33448 (Lakto 467) and Lactobacillus mucosae (Limosilactobacillus mucosae) (Limosilactobacillus mucosae (Limosilactobacillus mucosae)) DSM 33450 (Lakto 528) in the name of Novozymes Berlin GmbH, Gustav-Meyer-Allee 25, 13355 Berlin, Germany.

The present invention is therefore directed to a preparation comprising at least one probiotic strain selected from Lactobacillus helveticus DSM 33430, Lactobacillus rhamnosus (Lacticaseibacillus rhamnosus) DSM 33428, Lactobacillus mucosae (Limosilactobacillus mucosae) DSM 33450, and Lactobacillus johnsonii DSM 33448, and one or more anthocyanins.

In a preferred configuration of the present invention, the preparation comprises two or more of the listed probiotic strains, more preferred three or more of the probiotic strains and particularly preferred all the probiotic strains listed above.

More specifically, in a specific configuration for the production of ferulic acid, the preparation comprises Lactobacillus helveticus DSM 33430. In another specific configuration for the production of phloroglucinaldehyde the preparation comprises Lactobacillus rhamnosus (Lacticaseibacillus rhamnosus) DSM 33428. In another specific configuration for the production of protocatechuic acid the preparation comprises Lactobacillus mucosae (Limosilactobacillus mucosae) DSM 33450. In another specific configuration, the preparation comprises Lactobacillus johnsonii DSM 33448, which is able to produce high quantities of all three metabolites.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, and apples and vegetables such as red cabbage. Bilberries, in particular Vaccinium myrtillus, and black currants, in particular Ribes nigrum, are especially suitable.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but delphinidin- and cyanidin-3-O-glucoside (D3G, C3G) are also found (Gafner, Bilberry—Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

In an advantageous embodiment of the present invention anthocyanins are selected from cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

The anthocyanins can be from natural sources or from synthetic productions. Natural sources are preferably selected from fruits, flowers, leaves, stems and roots, preferably violet petal, seed coat of black soybean. Preferably anthocyanins are extracted from fruits selected from: acai, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin-3-rutinoside.

The counterion in the anthocyanins in the composition of the invention may be any physiologically tolerable counter anions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste. Besides the anthocyanins, the composition may desirably contain further beneficial or inactive ingredients, such as vitamins (preferably vitamin C), flavones, isoflavones, anticoagulants (e.g. maltodextrin, silica, etc.), desiccants, etc.

Another aspect of the invention is directed to a preparation which further comprising a targeted-release formulation for delayed release or enteric or colonic release. A targeted-release formulation according to the present invention is a formulation which ensures the delivery of the component of the preparation according to the present invention to a specific target in the body. A preferred formulation of such preparations promotes enteral or colonic delivery in the lower small intestine or in the large intestine. The targeted-release formulation can be obtained by adding enteric polymers to the matrix of the dosage form, or by adding a coating to the dosage form, preferably an enteric coating.

According to the present invention, a colon-specific delivery system is a delivery system, which targets the substance or drug directly to the colon. The advantage of a colon-specific delivery system is the local action, in case of disorders like ulcerative colitis, Crohn's disease, irritable bowel syndrome, and carcinomas. Targeted drug delivery to the colon in these cases ensures direct treatment at the site with lower dosing and fewer systemic side effects. In addition to local therapy colon can also be utilized as the portal entry of the drugs into systemic circulation for example molecules that are degraded/poorly absorbed in upper gut such as proteins and peptides may be better absorbed from the more benign environment of the colon. Colon-specific drug delivery is considered beneficial in the treatment of colon-related diseases and the oral delivery of protein and peptide drugs. Generally, each colon-specific drug delivery system has been designed based on one of the following mechanisms with varying degrees of success; 1. Coating with pH dependent polymers, 2. Coating with pH independent biodegradable polymers and 3. Delivery systems based on the metabolic activity of colonic bacteria.

An enteric coating is a barrier applied on oral medication that prevents its dissolution or disintegration in the gastric environment. Most enteric coatings work by presenting a surface that is stable at the intensely acidic pH found in the stomach but breaks down rapidly at a higher pH (alkaline pH). For example, they will not dissolve in the gastric acids of the stomach (pH ˜3), but they will start to dissolve in the environment present in the distal small intestine (pH range proximal to distal small intestine is ˜5.6 to 7.4) [11]. Colon targeted (drug) delivery systems are designed to selectively release a drug in response to the colonic environment without premature drug release in the upper GI tract.

The colon-specific delivery system can comprise a pH-dependent drug delivery system, since the colon exhibits a relatively higher pH than the upper GI tract. Accordingly, a colon-targeted delivery system is designed by using pH-dependent polymers such as cellulose acetate phthalates (CAP), hydroxypropyl methyl-cellulose phthalate (HPMCP) 50 and 55, copolymers of methacrylic acid and methyl methacrylate (e.g., Eudragit® S 100, Eudragit® L, Eudragit® FS, and Eudragit® P4135 F).

Therefore, in an advantageous configuration, the colon-specific delivery system comprises a coating comprising at least one pH dependent polymer or biodegradable polymer, preferably selected from methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein.

As a coating it is preferred to use a polymer polymerized from 10 to 30% by weight methyl methacrylate, 50 to 70% by weight methyl acrylate and 5 to 15% by weight methacrylic acid.

The polymer dispersion as disclosed may preferably comprise 15 to 50% by weight of a polymer polymerized from 20 to 30% by weight methyl methacrylate, 60 to 70% by weight methyl acrylate and 8 to 12% by weight methacrylic acid. Most preferred the polymer is polymerized from 25% by weight methyl methacrylate, 65% by weight methyl acrylate and 10% by weight methacrylic acid.

A 30% by weight aqueous dispersion of a polymer polymerized from 25% by weight methyl methacrylate, 65% by weight methyl acrylate and 10% by weight methacrylic acid corresponds to the commercial product EUDRAGUARD® biotic.

The percentages of the monomers add up to 100%. The functional polymer is applied in amounts of 2-30 mg/cm², preferably 5-20 mg/cm².

The Lactobacillus strains used for the preparations according to the present invention are selected from the following group:
a) One of the Lactobacillus strains as deposited under DSM 33430, DSM 33428, DSM 33450, or DSM 33448;
b) a mutant of the strain as deposited under DSM 33430 having all identifying characteristics of the strain DSM 33430, wherein said mutant preferably has a DNA sequence identity to the strain DSM 33430 of at least 95%, preferably at least 96, 97 or 98%, more preferably at least 99 or 99.5%;
c) a preparation of (a) or (b);
d) a preparation containing an effective mixture of metabolites as contained in (a), (b) or (c).

In a preferred configuration, the Lactobacillus helveticus strain DSM 33430 exhibits the following characterizing sequences:
a) A groL sequence with a sequence identity of at least 99.5%, above all 100%, to the polynucleotide sequence according to SEQ ID NO: 1;
b) A gyrB sequence with a sequence identity of at least 99.5%, above all 100%, to the polynucleotide sequence according to SEQ ID NO: 2;
c) A dnaA sequence with a sequence identity of at least 99.5%, above all 100%, to the polynucleotide sequence according to SEQ ID NO: 3;
d) A rpsK sequence with a sequence identity of at least 99.5%, above all 100%, to the polynucleotide sequence according to SEQ ID NO: 4;
e) A rpmB sequence with a sequence identity of at least 99.5%, above all 100%, to the polynucleotide sequence according to SEQ ID NO: 5;
f) A consensus 16 rDNA sequence with a sequence identity of at least 99.5%, above all 100%, to the polynucleotide sequence according to SEQ ID NO: 6;

In a preferred configuration, the Lactobacillus rhamnosus (Lacticaseibacillus rhamnosus) strain DSM 33428 exhibits the following characterizing sequences:
a) A groL sequence with a sequence identity of at least 99.5%, above all 100%, to the polynucleotide sequence according to SEQ ID NO: 7;
b) A gyrB sequence with a sequence identity of at least 99.5%, above all 100%, to the polynucleotide sequence according to SEQ ID NO: 8;
c) A dnaA sequence with a sequence identity of at least 99.5%, above all 100%, to the polynucleotide sequence according to SEQ ID NO: 9;
d) A rpsK sequence with a sequence identity of at least 99.5%, above all 100%, to the polynucleotide sequence according to SEQ ID NO: 10;
e) A rpmB sequence with a sequence identity of at least 99.5%, above all 100%, to the polynucleotide sequence according to SEQ ID NO: 11;
f) A consensus 16 rDNA sequence with a sequence identity of at least 99.5%, above all 100%, to the polynucleotide sequence according to SEQ ID NO: 12;
g) A prtP2 sequence with a sequence identity of at least 99.5%, above all 100%, to the polynucleotide sequence according to SEQ ID NO: 13;
h) A ohrR sequence with a sequence identity of at least 99.5%, above all 100%, to the polynucleotide sequence according to SEQ ID NO: 14;
i) A czcD sequence with a sequence identity of at least 99.5%, above all 100%, to the polynucleotide sequence according to SEQ ID NO: 15;

In a preferred configuration, the Lactobacillus mucosae (Limosilactobacillus mucosae) strain DSM 33450 exhibits the following characterizing sequences:
a) A groL sequence with a sequence identity of at least 99.5%, above all 100%, to the polynucleotide sequence according to SEQ ID NO: 16;
b) A gyrB sequence with a sequence identity of at least 99.5%, above all 100%, to the polynucleotide sequence according to SEQ ID NO: 17;
c) A dnaA sequence with a sequence identity of at least 99.5%, above all 100%, to the polynucleotide sequence according to SEQ ID NO: 18;
d) A rpsK sequence with a sequence identity of at least 99.5%, above all 100%, to the polynucleotide sequence according to SEQ ID NO: 19;
e) A rpmB sequence with a sequence identity of at least 99.5%, above all 100%, to the polynucleotide sequence according to SEQ ID NO: 20;
f) A consensus 16 rDNA sequence with a sequence identity of at least 99.5%, above all 100%, to the polynucleotide sequence according to SEQ ID NO: 21;

In a preferred configuration, the Lactobacillus johnsonii strain DSM 33448 exhibits the following characterizing sequences:
a) A rpsK sequence with a sequence identity of at least 99.5%, above all 100%, to the polynucleotide sequence according to SEQ ID NO: 22;
b) A rpsM sequence with a sequence identity of at least 99.5%, above all 100%, to the polynucleotide sequence according to SEQ ID NO: 23;
c) A rplB sequence with a sequence identity of at least 99.5%, above all 100%, to the polynucleotide sequence according to SEQ ID NO: 24;
d) A sdhA sequence with a sequence identity of at least 99.5%, above all 100%, to the polynucleotide sequence according to SEQ ID NO: 25;
e) A rpmB sequence with a sequence identity of at least 99.5%, above all 100%, to the polynucleotide sequence according to SEQ ID NO: 26;
f) A consensus 16s rDNA sequence with a sequence identity of at least 99.5%, above all 100%, to the polynucleotide sequence according to SEQ ID NO: 27;

Thus, a further subject of the current invention is a Lactobacillus strain, in particular a Lactobacillus strain as mentioned before, exhibiting at least one, preferably all of the following characteristics:
a) a groL sequence with a sequence identity of at least 99%, preferably at least 99.5%, more preferably at least 99.8 or 99.9%, above all 100%, to the polynucleotide sequence according to SEQ ID NO: 1 or SEQ ID NO: 7 or SEQ ID NO: 16;
c) a gyrB sequence with a sequence identity of at least 99%, preferably at least 99.5%, more preferably at least 99.8 or 99.9%, above all 100%, to the polynucleotide sequence according to SEQ ID NO: 2 or SEQ ID NO: 8 or SEQ ID NO: 17
d) a dnaA sequence with a sequence identity of at least 99%, preferably at least 99.5%, more preferably at least 99.8 or 99.9%, above all 100%, to the polynucleotide sequence according to SEQ ID NO: 3 or SEQ ID NO: 9 or SEQ ID NO: 18;
e) a rpsK sequence with a sequence identity of at least 99%, preferably at least 99.5%, more preferably at least 99.8 or 99.9%, above all 100%, to the polynucleotide sequence according to SEQ ID NO: 4 or SEQ ID NO: 10 or SEQ ID NO: 19.
f) a rpmB sequence with a sequence identity of at least 99%, preferably at least 99.5%, more preferably at least 99.8 or 99.9%, above all 100%, to the polynucleotide sequence according to SEQ ID NO: 5 or SEQ ID NO: 11 or SEQ ID NO: 20.
g) a 16S rDNA consensus sequence with a sequence identity of at least 99%, preferably at least 99.5%, more preferably at least 99.8 or 99.9%, above all 100%, to the polynucleotide sequence according to SEQ ID NO: 6 or SEQ ID NO: 12 or SEQ ID NO: 21.

A further subject of the current invention is a Lactobacillus strain, in particular a Lactobacillus strain as mentioned before, exhibiting at least one, preferably all of the following characteristics:
a) a rpsK sequence with a sequence identity of at least 99%, preferably at least 99.5%, more preferably at least 99.8 or 99.9%, above all 100%, to the polynucleotide sequence according to SEQ ID NO: 22.
b) a rpsM sequence with a sequence identity of at least 99%, preferably at least 99.5%, more preferably at least 99.8 or 99.9%, above all 100%, to the polynucleotide sequence according to SEQ ID NO: 23.
c) a rplB sequence with a sequence identity of at least 99%, preferably at least 99.5%, more preferably at least 99.8 or 99.9%, above all 100%, to the polynucleotide sequence according to SEQ ID NO: 24.
d) a sdhA sequence with a sequence identity of at least 99%, preferably at least 99.5%, more preferably at least 99.8 or 99.9%, above all 100%, to the polynucleotide sequence according to SEQ ID NO: 25.
e) a rpmB sequence with a sequence identity of at least 99%, preferably at least 99.5%, more preferably at least 99.8 or 99.9%, above all 100%, to the polynucleotide sequence according to SEQ ID NO: 26.
f) a 16S rDNA consensus sequence with a sequence identity of at least 99%, preferably at least 99.5%, more preferably at least 99.8 or 99.9%, above all 100%, to the polynucleotide sequence according to SEQ ID NO: 27.

A preferred formulation for colonic delivery of a preparation of this invention is a formulation that provides protection against gastric conditions as well as targeted release of the preparation in the large intestine. Therefore, in a preferred embodiment, the preparation comprises a coating for colonic release, applying coating materials preferably selected from methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein.

The preparation may further contain further carbohydrate ingredients, selected from arabinoxylans, barley grain fibre, oat grain fibre, rye fibre, wheat bran fibre, inulins, fructooligosaccharides (FOS), galactooligosaccharides (GOS), resistant starch, beta-glucans, glucomannans, galactoglucomannans, guar gum and xylooligosaccharides.

The preparation may further contain one or more plant extracts, selected from ginger, cinnamon, grapefruit, parsley, turmeric, curcuma, olive fruit, panax ginseng, horseradish, garlic, broccoli, spirulina, pomegranate, cauliflower, kale, cilantro, green tea, onions, and milk thistle.

The preparation may further contain astaxanthin, charcoal, chitosan, glutathione, monacolin K, plant sterols, plant stanols, sulforaphane, collagen, hyalurone, phosphatidylcholine.

The preparation may comprise further vitamins selected from biotin, vitamin A, vitamin B1 (thiamine), vitamin B2 (riboflavin), vitamin B3 (niacin), vitamin B5 (pantothenic acid), vitamin B9 (folic acid or folate), vitamin C (ascorbic acid), vitamin D (calciferols), vitamin E (tocopherols and tocotrienols) and vitamin K (quinones) or minerals selected from sulfur, iron, chlorine, calcium, chromium, cobalt, copper, magnesium, manganese, molybdenum, iodine, selenium, and zinc.

One subject of the present invention is the use of a preparation according to the present invention as food supplement or its use in foodstuffs. Preferred foodstuffs according to the invention are chocolate products, gummies, mueslis, muesli bars, dairy products, beverages, breads, pastries, cereal products.

A further subject of the current invention is also the use of a preparation of the current invention as food supplement, more specifically as synbiotic ingredient in food products.

A further subject of the present invention is a food supplement or foodstuff containing a preparation according to the present invention and at least one further feed or food ingredient, preferably selected from proteins, carbohydrates, fats, further probiotics, prebiotics, enzymes, vitamins, immune modulators, milk replacers, minerals, amino acids, coccidiostats, acid-based products, medicines, and combinations thereof.

The foodstuff composition according to the present invention does also include dietary supplements in the form of a pill, capsule, tablet or liquid.

The preparations according to the present invention, when administered to human beings, preferably improve the health status, in particular mental health, gut health, immune health, healthy weight of a human being.

A further subject of the current invention is therefore a composition according to the present invention for improving the health status, in particular mental health, gut health, immune health, healthy weight of a human being.

Alternatively, the composition according to the present invention may be suitable for use in the prevention and treatment of metabolic disorders, hypertension, prediabetes, diabetes, and Alzheimer's disease, atherosclerosis, cancer, high cholesterol, menopause symptoms, osteoporosis.

An advantageous configuration according to the present invention is a composition for improving the health status of an animal or a human being by increasing the total amount of the following AC metabolites in the host, more specifically ferulic acid, protocatechuic acid and phloroglucinaldehyde.

### WORKING EXAMPLES

The berry extracts composition (Healthberry® 865; Evonik Nutrition & Care GmbH, Darmstadt, Germany) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The relative content of each anthocyanin in the Healthberry® 865 product was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin;

2.5% of 3-O-b-glucosides, 3-O-b-galactosides, and 3-O-b-arabinosides of petunidin; 2.5% of 3-O-b-glucosides, 3-O-b-galactosides, and 3-O-b-arabinosides of peonidin; 3.0% of 3-O-b-glucosides, 3-0-b-galactosides, and 3-O-b-arabinosides of malvidin.

The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

### Example 1: Lactobacillus Species Display Varying Potential of Producing AC Metabolites

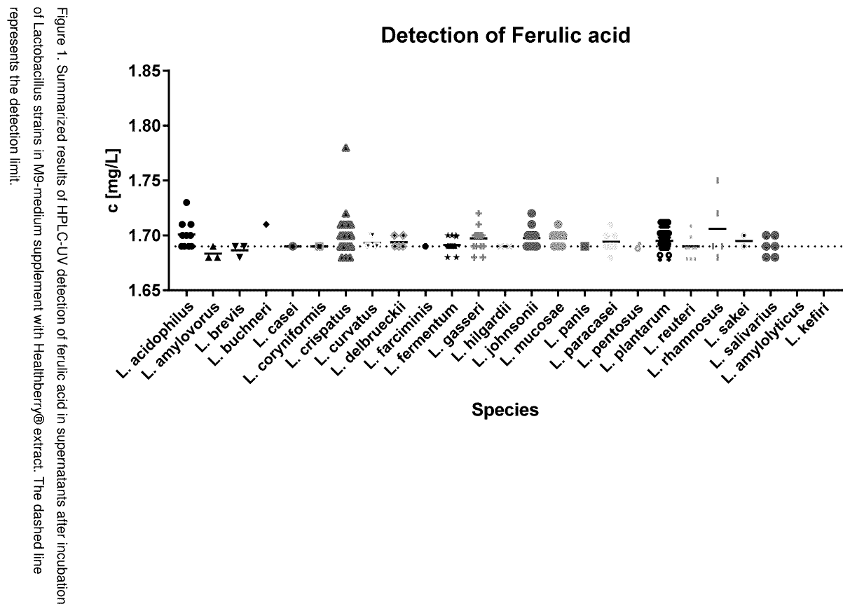

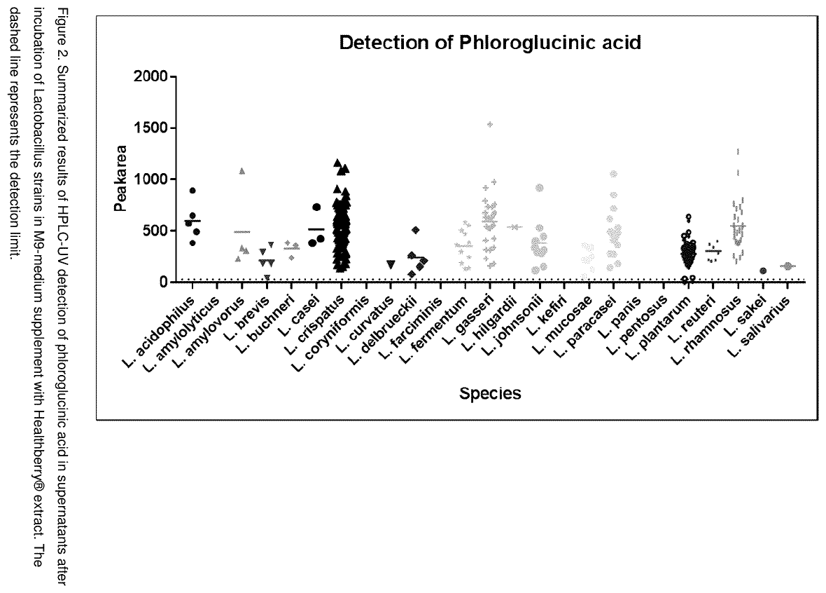

600 different Lactobacillus strains were cultivated under anaerobic conditions in microtiter plates with MRS medium for 48 h at 37° C. Afterwards the cells were harvested by centrifugation at 4000×g for 10 min and washed with PBS buffer. Subsequently the cells were resuspended in M9-medium adjusted to pH 6.0 and supplemented with 115 mg/I Healthberry® (an extract from bilberry and blackcurrant with standardized anthocyan content) and transferred to deep-well plates. After 6 h incubation under anaerobic conditions at 37° C., the cells were removed by centrifugation at 4000×g for 10 min. Product formation was determined by HPLC analysis of the supernatant. The results are displayed in FIGS. 1-4.

FIG. 1 shows the summarized results of HPLC-UV detection of ferulic acid in supernatants after incubation of Lactobacillus strains in M9-medium supplement with Healthberry® extract. The dashed line represents the detection limit.

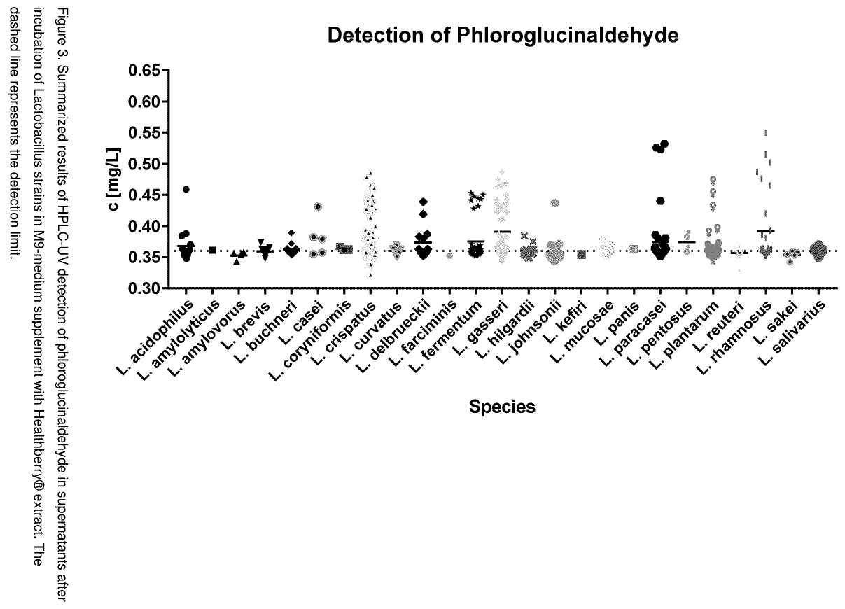

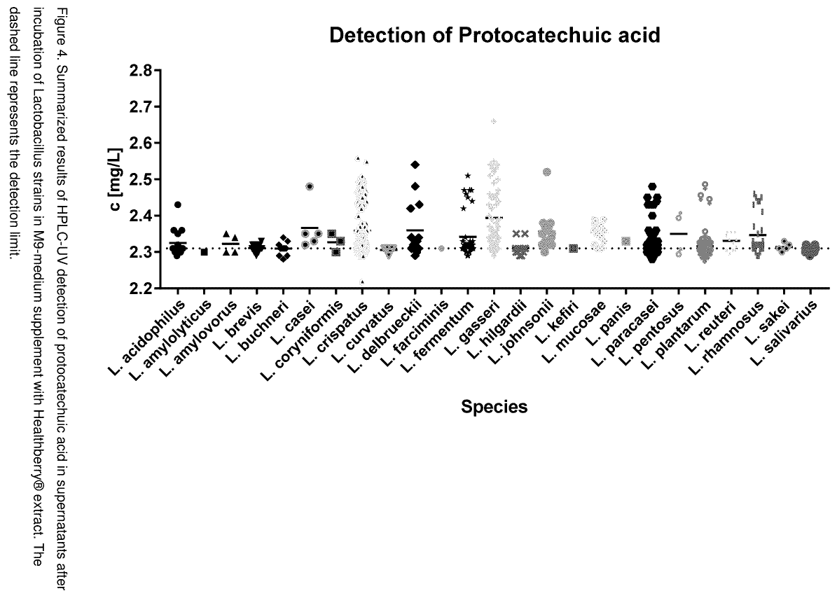

FIG. 2 shows the summarized results of HPLC-UV detection of phloroglucinic acid in supernatants after incubation of Lactobacillus strains in M9-medium supplement with Healthberry® extract. The dashed line represents the detection limit.

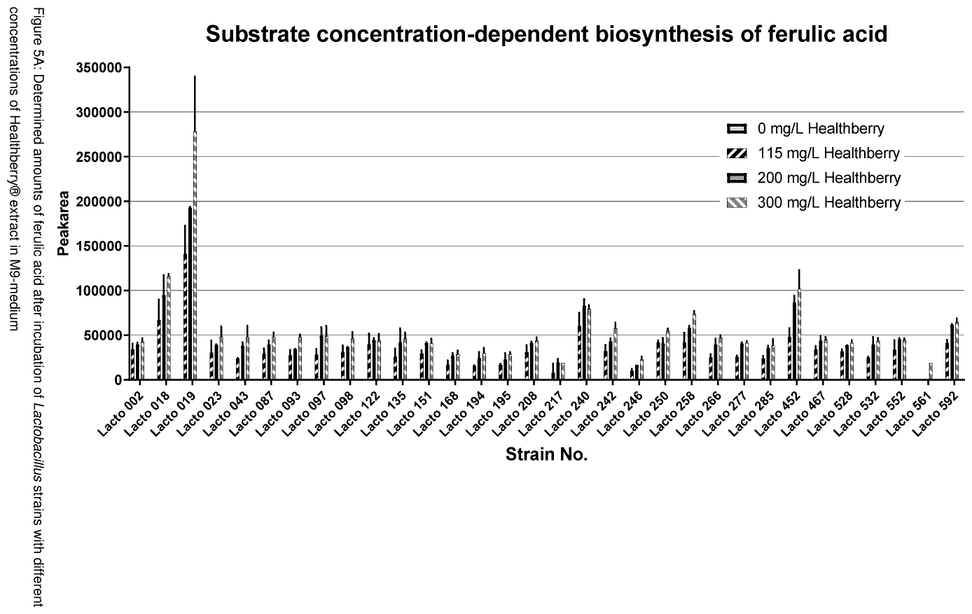

FIG. 3 shows the summarized results of HPLC-UV detection of phloroglucinaldehyde in supernatants after incubation of Lactobacillus strains in M9-medium supplement with Healthberry® extract. The dashed line represents the detection limit.

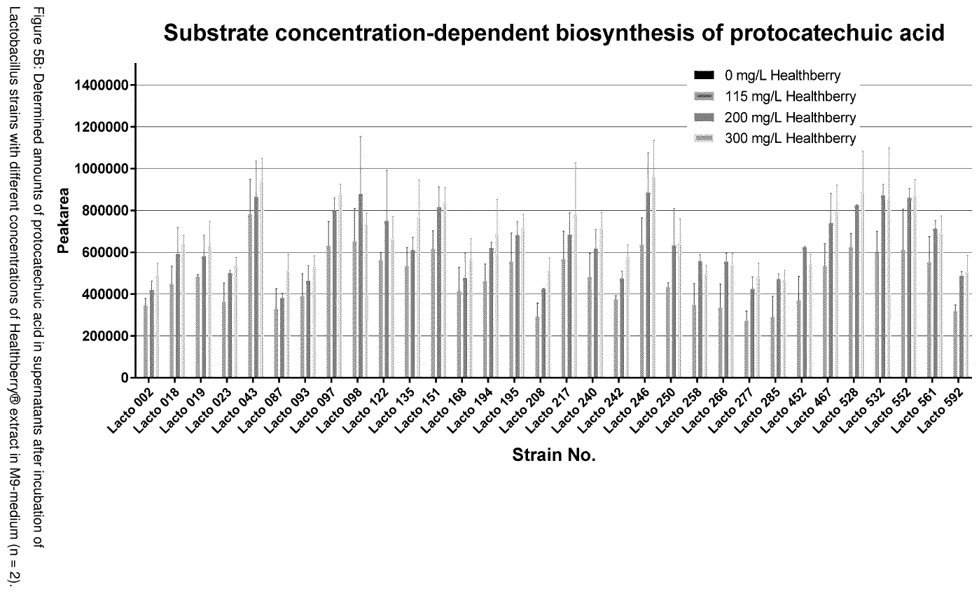

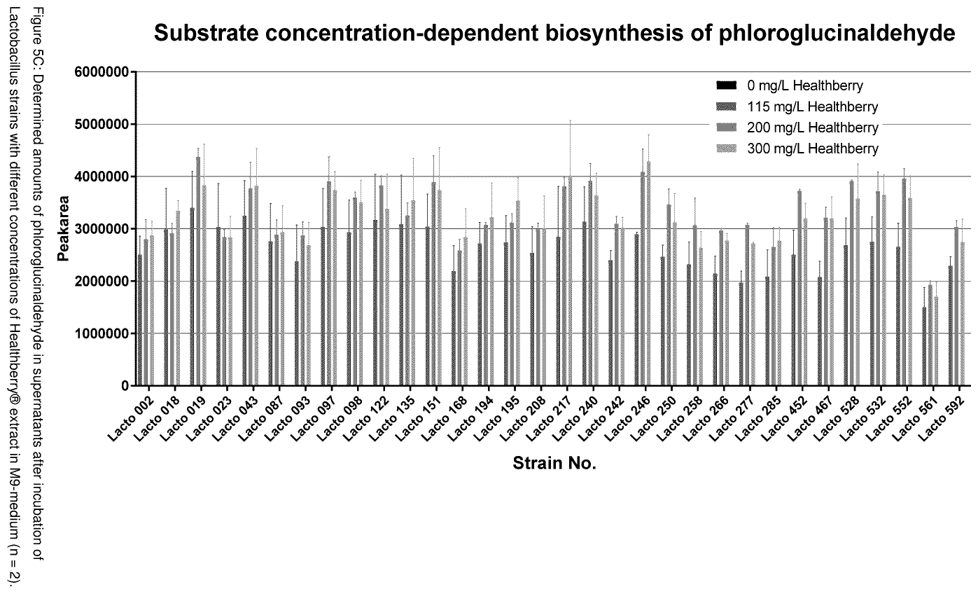

FIG. 4 shows the summarized results of HPLC-UV detection of protocatechuic acid in supernatants after incubation of Lactobacillus strains in M9-medium supplement with Healthberry® extract. The dashed line represents the detection limit.

The potential to produce AC metabolites is species- and strain-specific (as displayed in FIGS. 1-4). Importantly, further analysis revealed that some strains produce primarily one of the metabolites analysed, whereas others produce multiple metabolites in relatively high amounts.

### Example 2: AC Metabolite Spectra Produced by Lactobacillus Strains

Lactobacillus strains were assessed for AC metabolite production using Healthberry® as substrate as detailed in example 2. A rating system for metabolite production was applied to highlight strains that facilitate production of one or more than one AC metabolites. For the selection of best strains for subsequent analyses the best producers of single metabolites as well as the best allround producers were selected. Therefore, a rating system considering all four time points for each compound has been applied in which up to 3 points were scored for each compound depending on the individual productivity of each strain related to the intermediate productivity of all strains tested. 3 points were scored when a strain produces more than 130% of the intermediate concentration of all strains. 2 points when a strain produces more than 120% and 1 point when a strain produces more than 110% of the intermediate value. For selection of 10 candidates Top 2 strains for each compound detected (3×2=6) and best 4 allround strains have been selected as shown in table 1. Lakto 019 as Lactobacillus helveticus DSM 33430, Lakto 018 as Lactobacillus rhamnosus (Lacticaseibacillus rhamnosus) DSM 33428, Lakto 528 as Lactobacillus mucosae (Limosilactobacillus mucosae) DSM 33450, Lakto 467 as Lactobacillus johnsonii DSM 33448 were selected as the best performer according to this rating system. More specifically, Lakto 019 as Lactobacillus helveticus DSM 33430 was selected as best producer of ferulic acid, Lakto 018 as Lactobacillus rhamnosus (Lacticaseibacillus rhamnosus) DSM 33428 was selected as best producer of phloroglucinaldehyde, Lakto 528 as Lactobacillus mucosae (Limosilactobacillus mucosae) DSM 33450 was selected as best producer of protocatechuic acid and Lakto 467 as Lactobacillus johnsonii DSM 33448 was selected as best allround strain, being able to produce high quantities of all three metabolites.

TABLE 1
Production of AC metabolites production using Healthberry ® as
substrate in different Lactobacillus strains

| | | Protocatechuic | Phloroglucin- | Ferulic | |
|---|---|---|---|---|---|
| Strain No. | Species | acid | aldehyde | acid | Sum |
| Lacto 018 | Lactobacillus rhamnosus | 3 | 12 | 6 | 21 |
| Lacto 019 | Lactobacillus acidophilus | 3 | 4 | 12 | 19 |
| Lacto 043 | Lactobacillus delbrueckii | 1 | 7 | 0 | 8 |
| | subsp. delbrueckii | | | | |
| Lacto 097 | Lactobacillus mucosae | 9 | 7 | 0 | 16 |
| Lacto 098 | Lactobacillus mucosae | 7 | 5 | 0 | 12 |
| Lacto 122 | Lactobacillus mucosae | 3 | 1 | 0 | 4 |
| Lacto 135 | Lactobacillus gasseri | 1 | 2 | 0 | 3 |
| Lacto 151 | Lactobacillus delbrueckii | 0 | 4 | 0 | 4 |
| | subsp. delbrueckii | | | | |
| Lacto 217 | Lactobacillus gasseri | 0 | 3 | 0 | 3 |
| Lacto 240 | Lactobacillus crispatus | 0 | 3 | 0 | 3 |
| Lacto 246 | Lactobacillus gasseri | 4 | 4 | 0 | 8 |
| Lacto 250 | Lactobacillus crispatus | 4 | 8 | 0 | 12 |
| Lacto 258 | Lactobacillus crispatus | 0 | 1 | 3 | 4 |
| Lacto 452 | Lactobacillus plantarum | 3 | 0 | 3 | 6 |
| | subsp. argentoratensis | | | | |
| Lacto 467 | Lactobacillus johnsonii | 7 | 9 | 10 | 26 |
| Lacto 528 | Lactobacillus mucosae | 9 | 3 | 0 | 12 |
| Lacto 532 | Lactobacillus mucosae | 9 | 1 | 0 | 10 |
| Lacto 552 | Lactobacillus mucosae | 7 | 4 | 0 | 11 |
| Lacto 561 | Lactobacillus johnsonii | 7 | 1 | 3 | 11 |
| Lacto 592 | Lactobacillus plantarum | 0 | 3 | 2 | 5 |

We observed that the average production of ferulic acid was higher within the Lactobacillus plantarum and Lactobacillus helveticus species as compared to others. When comparing a large number of strains of both species, we observed that the strains Lactobacillus rhamnosus DSM 33428 and Lactobacillus helveticus DSM 33430 produced surprisingly high amounts of ferulic acid, exceeding all other tested strains by on average more than 400% and 250% and the next best alternative strain by 195% and 100%, respectively (see table 2).

Likely, protocatechuic acid was prominent for Lactobacillus mucosae, and we discovered that the strain Lactobacillus mucosae DSM 33450 stands out against other strains of this species by exceeding the average production of this metabolite by 39%, and the next best alternative by 15% (see table 3).

TABLE 2
Production of ferulic acid by Lactobacillus plantarum
and Lactobacillus helveticus species. AUC (area under
the curve) values were retrieved from HPLC analyses and
correspond to metabolite concentration levels.

| | | Ferulic |
|---|---|---|
| Strain ID | Species | acid AUC |
| Lacto 18 (DSM 33428) | Lactobacillus rhamnosus | 2660639 |
| Lacto 19 (DSM 33430) | Lactobacillus helveticus | 1805488 |
| Lacto 29 | Lactobacillus rhamnosus | 901757 |
| Lacto 311 | Lactobacillus rhamnosus | 753191 |
| Lacto 297 | Lactobacillus rhamnosus | 721643 |
| Lacto 41 | Lactobacillus rhamnosus | 679032 |
| Lacto 205 | Lactobacillus rhamnosus | 524701 |
| Lacto 155 | Lactobacillus rhamnosus | 472779 |
| Lacto 42 | Lactobacillus rhamnosus | 462981 |
| Lacto 118 | Lactobacillus rhamnosus | 448989 |
| Lacto 37 | Lactobacillus rhamnosus | 439027 |
| Lacto 72 | Lactobacillus rhamnosus | 425825 |
| Lacto 39 | Lactobacillus rhamnosus | 400168 |
| Lacto 188 | Lactobacillus rhamnosus | 398403 |
| Lacto 424 | Lactobacillus rhamnosus | 387779 |
| Lacto 14 | Lactobacillus rhamnosus | 375532 |
| Lacto 154 | Lactobacillus rhamnosus | 328846 |
| Lacto 01 | Lactobacillus rhamnosus | 327641 |
| Lacto 161 | Lactobacillus rhamnosus | 308091 |
| Lacto 44 | Lactobacillus rhamnosus | 276178 |
| Lacto 092 | Lactobacillus rhamnosus | 268607 |
| Lacto 196 | Lactobacillus rhamnosus | 267739 |
| Lacto 223 | Lactobacillus rhamnosus | 263916 |
| Lacto 225 | Lactobacillus rhamnosus | 258761 |
| Lacto 578 | Lactobacillus rhamnosus | 258506 |
| Lacto 218 | Lactobacillus rhamnosus | 232534 |
| Lacto 197 | Lactobacillus rhamnosus | 208713 |
| Lacto 579 | Lactobacillus rhamnosus | 195623 |
| Lacto 376 | Lactobacillus rhamnosus | 184252 |
| Lacto 336 | Lactobacillus rhamnosus | 141135 |

TABLE 3
Production of protocatechuic acid by Lactobacillus mucosae species.
AUC (area under the curve) values were retrieved from HPLC analyses
and correspond to metabolite concentration levels.

| | | Protocatechuic |
|---|---|---|
| Strain ID | Species | acid AUC |
| Lacto 528 (DSM 33450) | Lactobacillus mucosae | 14842754 |
| Lacto 497 | Lactobacillus mucosae | 12918819 |
| Lacto 098 | Lactobacillus mucosae | 12633741 |
| Lacto 122 | Lactobacillus mucosae | 11010992 |
| Lacto 123 | Lactobacillus mucosae | 8582625 |
| Lacto 278 | Lactobacillus mucosae | 7585482 |
| Lacto 127 | Lactobacillus mucosae | 6994715 |

### Example 3: Dose-Dependency and Kinetics of Ferulic Acid Production by Lactobacillus helveticus DSM 33430 (Lakto 019)

The assessment of dose- and time-dependency of ferulic acid production by several Lactobacillus strains revealed that dose-dependency was particularly prominent for Lakto 019 (Lactobacillus helveticus DSM 33430) (FIG. 5); moreover, AC were rapidly metabolized by this strain with peak levels occurring already after three hours incubation. These characteristics set Lakto 019 apart from all other strains tested and reported in the literature.

FIG. 5A shows the determined amounts of ferulic acid after incubation of Lactobacillus strains with different concentrations of Healthberry® extract in M9-medium.

FIG. 5B shows determined amounts of protocatechuic acid in supernatants after incubation of Lactobacillus strains with different concentrations of Healthberry® extract in M9-medium (n=2).

FIG. 5C shows determined amounts of phloroglucinaldehyde in supernatants after incubation of Lactobacillus strains with different concentrations of Healthberry® extract in M9-medium (n=2).

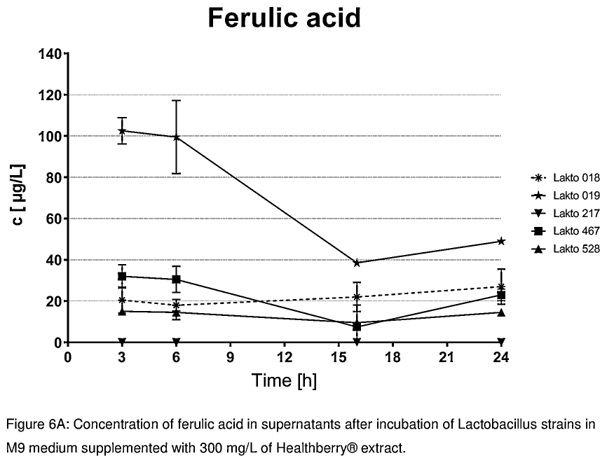

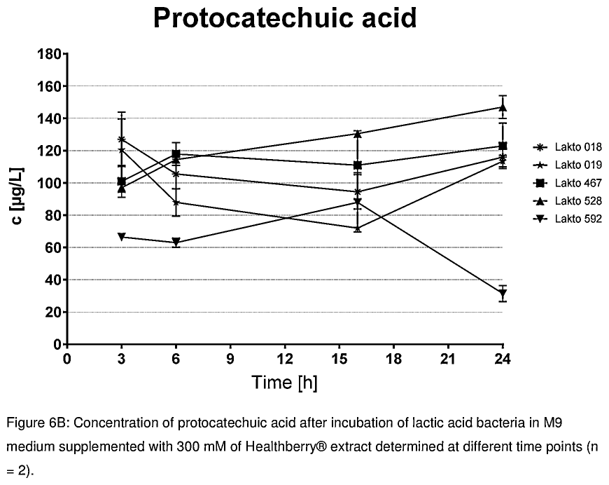

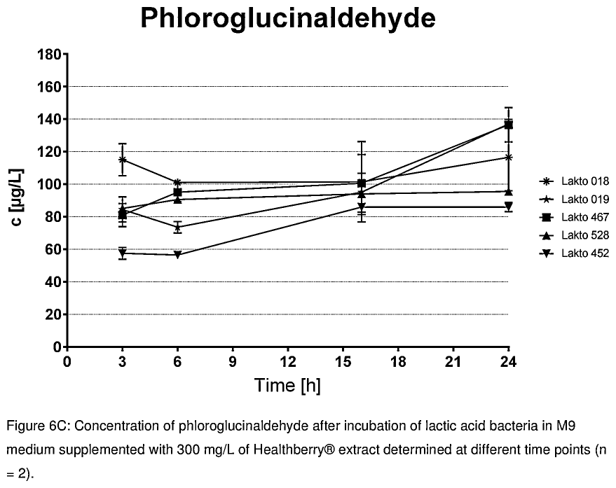

FIG. 6A shows the concentration of ferulic acid in supernatants after incubation of Lactobacillus strains in M9 medium supplemented with 300 mg/L of Healthberry® extract.

FIG. 6B shows concentration of protocatechuic acid after incubation of lactic acid bacteria in M9 medium supplemented with 300 mM of Healthberry® extract determined at different time points (n=2).

FIG. 6C shows concentration of phloroglucinaldehyde after incubation of lactic acid bacteria in M9 medium supplemented with 300 mg/L of Healthberry® extract determined at different time points (n=2).

### Example 4: Composition of Synbiotic Capsules

Capsules containing AC (or sources thereof), Lactobacillus spp. in different ratios, and various other ingredients. Table 4 shows preparations for filling into HPMC capsules.

TABLE 4
Preparations for filling into HPMC capsules. AC
may be added as mixture or as single compounds.

| Compound | Capsule I | Capsule II | Capsule III |
|---|---|---|---|
| Anthocyan mixture* | 250 mg | 50 mg | 800 mg |
| Lactobacillus strain^{#} | 1 × 10⁷ CFU- | 1 × 10⁷ CFU- | 1 × 10⁷ CFU- |
| | 1 × 10¹¹ CFU | 1 × 10¹¹ CFU | 1 × 10¹¹ CFU |

^{#}Strain selected from Lakto 019 as Lactobacillus helveticus DSM 33430, Lakto 018 as Lactobacillus rhamnosus (Lacticaseibacillus rhamnosus) DSM 33428, Lakto 528 as Lactobacillus mucosae (Limosilactobacillus mucosae) DSM 33450, Lakto 467 as Lactobacillus johnsonii DSM 33448.

### Example 5: Capsules Coated with Eudraguard® Biotic

HPMC capsules (size 3) were filled with a composition as described in table 2. The total capsule weight was 200 mg. The capsules were coated with an enteric coating composition as shown in table 5.

TABLE 5
Coating composition

| | | Content | | Content |
|---|---|---|---|---|
| | Dry | based on | Weight | based on |
| | substance | coating | gain | capsule |
| Compound | [g] | [%] | [%] | [%] |
| EUDRAGUARD ® | 40.8 | 36.9 | 8.2 | 6.7 |
| biotic | | | | |
| HPMC | 43.1 | 39.0 | 8.6 | 7.1 |
| Talc | 20.4 | 18.4 | 4.0 | 3.3 |
| Polyethylene | 4.3 | 3.9 | 0.9 | 0.7 |
| glycol | | | | |
| Triethyl | 2.0 | 1.8 | 0.4 | 0.3 |
| citrate | | | | |

### REFERENCES

1. Gibson G R, Hutkins R, Sanders M E, Prescott S L, Reimer R A, Salminen S J, Scott K, Stanton C, Swanson K S, Cani P D et al: Expert consensus document: The International Scientific Association for Probiotics and Prebiotics (ISAPP) consensus statement on the definition and scope of prebiotics. Nat Rev Gastroenterol Hepatol 2017, 14(8):491-502.
2. Hill C, Guarner F, Reid G, Gibson G R, Merenstein D J, Pot B, Morelli L, Canani R B, Flint H J, Salminen S et al: Expert consensus document. The International Scientific Association for Probiotics and Prebiotics consensus statement on the scope and appropriate use of the term probiotic. Nat Rev Gastroenterol Hepatol 2014, 11(8):506-514.
3. Pandey K R, Naik S R, Vakil B V: Probiotics, prebiotics and synbiotics—a review. J Food Sci Technol 2015, 52(12):7577-7587.
4. Hassellund S S, Flaa A, Kjeldsen S E, Seljeflot I, Karlsen A, Erlund I, Rostrup M: Effects of anthocyanins on cardiovascular risk factors and inflammation in pre-hypertensive men: a double-blind randomized placebo-controlled crossover study. J Hum Hypertens 2013, 27(2):100-106.
5. Kent K, Charlton K E, Netzel M, Fanning K: Food-based anthocyanin intake and cognitive outcomes in human intervention trials: a systematic review. J Hum Nutr Diet 2017, 30(3):260-274.
6. Liu Y, Li D, Zhang Y, Sun R, Xia M: Anthocyanin increases adiponectin secretion and protects against diabetes-related endothelial dysfunction. Am J Physiol Endocrinol Metab 2014, 306(8):E975-988.
7. Vendrame S, Del Bo C, Ciappellano S, Riso P, Klimis-Zacas D: Berry Fruit Consumption and Metabolic Syndrome. Antioxidants (Basel) 2016, 5(4).
8. Gibney E R, Milenkovic D, Combet E, Ruskovska T, Greyling A, Gonzalez-Sarrias A, de Roos B, Tomas-Barberan F, Morand C, Rodriguez-Mateos A: Factors influencing the cardiometabolic response to (poly)phenols and phytosterols: a review of the COST Action POSITIVe activities. Eur J Nutr 2019, 58(Suppl 2):37-47.
9. de Ferrars R M, Czank C, Zhang Q, Botting N P, Kroon P A, Cassidy A, Kay C D: The pharmacokinetics of anthocyanins and their metabolites in humans. Br J Pharmacol 2014, 171(13):3268-3282.
10. Cardona F, Andres-Lacueva C, Tulipani S, Tinahones F J, Queipo-Ortuno M I: Benefits of polyphenols on gut microbiota and implications in human health. J Nutr Biochem 2013, 24(8):1415-1422.
11. Hanske L, Engst W, Loh G, Sczesny S, Blaut M, Braune A: Contribution of gut bacteria to the metabolism of cyanidin 3-glucoside in human microbiota-associated rats. Br J Nutr 2013, 109(8):1433-1441.
12. Feliciano R P, Istas G, Heiss C, Rodriguez-Mateos A: Plasma and Urinary Phenolic Profiles after Acute and Repetitive Intake of Wild Blueberry. Molecules 2016, 21(9).
13. Kay C D, Pereira-Caro G, Ludwig I A, Clifford M N, Crozier A: Anthocyanins and Flavanones Are More Bioavailable than Previously Perceived: A Review of Recent Evidence. Annu Rev Food Sci Technol 2017, 8:155-180.
14. Braune A, Blaut M: Bacterial species involved in the conversion of dietary flavonoids in the human gut. Gut Microbes 2016, 7(3):216-234.
15. Cheng J R, Liu X M, Chen Z Y, Zhang Y S, Zhang Y H: Mulberry anthocyanin biotransformation by intestinal probiotics. Food Chem 2016, 213:721-727.
16. Mahmoud A M, Hussein O E, Hozayen W G, Bin-Jumah M, Abd El-Twab S M: Ferulic acid prevents oxidative stress, inflammation, and liver injury via upregulation of Nrf2/HO-1 signaling in methotrexate-induced rats. Environ Sci Pollut Res Int 2019.
17. E I-Sonbaty Y A, Suddek G M, Megahed N, Gameil N M: Protocatechuic acid exhibits hepatoprotective, vasculoprotective, antioxidant and insulin-like effects in dexamethasone-induced insulin-resistant rats. Biochimie 2019, 167:119-134.

### NUCLEOTIDE SEQUENCES

SEQ ID NO:1 Lactobacillus helveticus DSM 33430 groL
SEQ ID NO: 2 Lactobacillus helveticus DSM 33430 gyrB
SEQ ID NO: 3 Lactobacillus helveticus DSM 33430 dnaA
SEQ ID NO: 4 Lactobacillus helveticus DSM 33430 rpsK
SEQ ID NO: 5 Lactobacillus helveticus DSM 33430 rpmB
SEQ ID NO: 6 Lactobacillus helveticus DSM 33430 consensus 16 rDNA
SEQ ID NO: 7 Lactobacillus rhamnosus (Lacticaseibacillus rhamnosus) DSM 33428 groL
SEQ ID NO: 8 Lactobacillus rhamnosus (Lacticaseibacillus rhamnosus) DSM 33428 gyrB
SEQ ID NO: 9 Lactobacillus rhamnosus (Lacticaseibacillus rhamnosus) DSM 33428 dnaA
SEQ ID NO: 10 Lactobacillus rhamnosus (Lacticaseibacillus rhamnosus) DSM 33428 rpsK
SEQ ID NO: 11 Lactobacillus rhamnosus (Lacticaseibacillus rhamnosus) DSM 33428 rpmB
SEQ ID NO: 12 Lactobacillus rhamnosus (Lacticaseibacillus rhamnosus) DSM 33428 16rDNA
SEQ ID NO: 13 Lactobacillus rhamnosus (Lacticaseibacillus rhamnosus) DSM 33428 prtP2
SEQ ID NO: 14 Lactobacillus rhamnosus (Lacticaseibacillus rhamnosus) DSM 33428 ohrR
SEQ ID NO: 15 Lactobacillus rhamnosus (Lacticaseibacillus rhamnosus) DSM 33428 czcD
SEQ ID NO: 16 Lactobacillus mucosae (Limosilactobacillus mucosae) DSM 33450 groL
SEQ ID NO: 17 Lactobacillus mucosae (Limosilactobacillus mucosae) DSM 33450 gyrB
SEQ ID NO: 18 Lactobacillus mucosae (Limosilactobacillus mucosae) DSM 33450 dnaA
SEQ ID NO: 19 Lactobacillus mucosae (Limosilactobacillus mucosae) DSM 33450 rpsK
SEQ ID NO: 20 Lactobacillus mucosae (Limosilactobacillus mucosae) DSM 33450 rpmB
SEQ ID NO: 21 Lactobacillus mucosae (Limosilactobacillus mucosae) DSM 33450 consensus 16 rDNA
SEQ ID NO: 22 Lactobacillus johnsonii DSM 33448 rpsK
SEQ ID NO: 23 Lactobacillus johnsonii DSM 33448 rpsM
SEQ ID NO: 24 Lactobacillus johnsonii DSM 33448 rplB
SEQ ID NO: 25 Lactobacillus johnsonii DSM 33448 sdhA
SEQ ID NO: 26 Lactobacillus johnsonii DSM 33448 rpmB
SEQ ID NO: 27 Lactobacillus johnsonii DSM 33448 consensus 16s rDNA

1-13. (canceled)

14. A preparation comprising:
at least one probiotic strain selected from the group consisting of: Lactobacillus helveticus DSM 33430; Lactobacillus rhamnosus (Lacticaseibacillus rhamnosus) DSM 33428; Lactobacillus mucosae (Limosilactobacillus mucosae) DSM 33450; and Lactobacillus johnsonii DSM 33448; and
one or more anthocyanins.

15. The preparation of claim 14, wherein the anthocyanins are selected from the group consisting of: cyanidin-3-glucoside; cyanidin-3-galactoside; cyanidin-3-arabinoside; delphinidin-3-glucoside; delphinidin-3-galactoside; delphinidin-3-arabinoside; petunidin-3-glucoside; petunidin-3-galactoside; petunidin-3-arabinose; peonidin-3-glucoside; peonidin-3-galactoside; peonidin-3-arabinose; malvidin-3-glucoside; malvidin-3-galactoside; malvidin-3-arabinose; cyanidin-3-rutinoside; and delphinidin-3-rutinoside.

16. The preparation of claim 14, wherein the anthocyanins are selected from the group consisting of: cyanidin-3-glucoside; cyanidin-3-rutinoside; delphinidin-3-glucoside; delphinidin-3-rutinoside; cyanidin-3-galactoside; and delphinidin-3-galactoside.

17. The preparation of claim 14, wherein the anthocyanins are in a fruit or plant extract at a concentration of at least 5 weight-%.

18. The preparation of claim 17, wherein the anthocyanins are in a fruit or plant extract at a concentration of at least 25 weight-%.

19. The preparation of claim 14, further comprising a targeted-release formulation for delayed release, enteric release or colonic release.

20. The preparation of claim 19, wherein the targeted-release formulation comprises a coating comprising at least one pH dependent polymer or biodegradable polymer.

21. The preparation of claim 20, wherein the coating comprises methyl acrylate-methacrylic acid copolymers; cellulose acetate phthalate (CAP); cellulose acetate succinate; hydroxypropyl methyl cellulose phthalate; hydroxypropyl methyl cellulose acetate succinate (hypromellose acetate succinate); polyvinyl acetate phthalate (PVAP); methyl methacrylate-methacrylic acid copolymers; shellac; cellulose acetate trimellitate; sodium alginate; or zein.

22. The preparation of claim 20, wherein the coating comprises a polymer polymerized from 10 to 30% by weight methyl methacrylate, 50 to 70% by weight methyl acrylate and to 15% by weight methacrylic acid.

23. The preparation of claim 20, wherein the coating comprises 15 to 50% by weight of a polymer polymerized from 20 to 30% by weight methyl methacrylate, 60 to 70% by weight methyl acrylate and 8 to 12% by weight methacrylic acid.

24. The preparation of claim 14, wherein the probiotic strain is Lactobacillus helveticus DSM 33430 and exhibits the following characteristics:
a) a groL sequence with a sequence identity of at least 99.5% to the polynucleotide sequence of SEQ ID NO: 1;
b) a gyrB sequence with a sequence identity of at least 99.5% to the polynucleotide sequence of SEQ ID NO: 2;
c) a dnaA sequence with a sequence identity of at least 99.5% to the polynucleotide sequence of SEQ ID NO: 3;
d) a rpsK sequence with a sequence identity of at least 99.5% to the polynucleotide sequence of SEQ ID NO: 4;
e) a rpmB sequence with a sequence identity of at least 99.5% to the polynucleotide sequence of SEQ ID NO: 5;
f) a consensus 16 rDNA sequence with a sequence identity of at least 99.5% to the polynucleotide sequence of SEQ ID NO: 6.

25. The preparation of claim 14, wherein the probiotic strain is Lactobacillus helveticus DSM 33430 and exhibits the following characteristics:
a) a groL sequence with a sequence identity of 100% to the polynucleotide sequence of SEQ ID NO: 1;
b) a gyrB sequence with a sequence identity of 100% to the polynucleotide sequence of SEQ ID NO: 2;
c) a dnaA sequence with a sequence identity of 100% to the polynucleotide sequence of SEQ ID NO: 3;
d) a rpsK sequence with a sequence identity of 100% to the polynucleotide sequence of SEQ ID NO: 4;
e) a rpmB sequence with a sequence identity of 100% to the polynucleotide sequence of SEQ ID NO: 5;
f) a consensus 16 rDNA sequence with a sequence identity of 100% to the polynucleotide sequence of SEQ ID NO: 6.

26. The preparation of claim 14, wherein the probiotic strain is Lactobacillus rhamnosus DSM 33428 and exhibits the following characteristics:
a) a groL sequence with a sequence identity of at least 99.5% to the polynucleotide sequence of SEQ ID NO: 7;
b) a gyrB sequence with a sequence identity of at least 99.5% to the polynucleotide sequence of SEQ ID NO: 8;
c) a dnaA sequence with a sequence identity of at least 99.5% to the polynucleotide sequence of SEQ ID NO: 9;
d) a rpsK sequence with a sequence identity of at least 99.5% to the polynucleotide sequence of SEQ ID NO: 10;
e) a rpmB sequence with a sequence identity of at least 99.5% to the polynucleotide sequence of SEQ ID NO: 11;
f) a consensus 16 rDNA sequence with a sequence identity of at least 99.5% to the polynucleotide sequence of SEQ ID NO: 12;
g) a prtP2 sequence with a sequence identity of at least 99.5% to the polynucleotide sequence of SEQ ID NO: 13;
h) an ohrR sequence with a sequence identity of at least 99.5% to the polynucleotide sequence of SEQ ID NO: 14;
i) a czcD sequence with a sequence identity of at least 99.5% to the polynucleotide sequence of SEQ ID NO: 15.

27. The preparation of claim 14, wherein the probiotic strain is Lactobacillus rhamnosus DSM 33428 and exhibits the following characteristics:
a) a groL sequence with a sequence identity of 100% to the polynucleotide sequence of SEQ ID NO: 7;
b) a gyrB sequence with a sequence identity of 100% to the polynucleotide sequence of SEQ ID NO: 8;
c) a dnaA sequence with a sequence identity of 100% to the polynucleotide sequence of SEQ ID NO: 9;
d) a rpsK sequence with a sequence identity of 100% to the polynucleotide sequence of SEQ ID NO: 10;
e) a rpmB sequence with a sequence identity of 100% to the polynucleotide of SEQ ID NO: 11;
f) a consensus 16 rDNA sequence with a sequence identity of 100% to the polynucleotide sequence of SEQ ID NO: 12;
g) a prtP2 sequence with a sequence identity of 100% to the polynucleotide sequence of SEQ ID NO: 13;
h) an ohrR sequence with a sequence identity of 100% to the polynucleotide sequence of SEQ ID NO: 14;
i) a czcD sequence with a sequence identity of 100% to the polynucleotide sequence of SEQ ID NO: 15.

28. The preparation of claim 14, wherein the probiotic strain is Lactobacillus mucosae (Limosilactobacillus mucosae) DSM 33450 and exhibits the following characteristics:
a) a groL sequence with a sequence identity of at least 99.5% to the polynucleotide sequence of SEQ ID NO: 16;
b) a gyrB sequence with a sequence identity of at least 99.5% to the polynucleotide sequence of SEQ ID NO: 17;
c) a dnaA sequence with a sequence identity of at least 99.5% to the polynucleotide sequence of SEQ ID NO: 18;
d) a rpsK sequence with a sequence identity of at least 99.5% to the polynucleotide sequence of SEQ ID NO: 19;
e) a rpmB sequence with a sequence identity of at least 99.5% to the polynucleotide sequence of SEQ ID NO: 20;
f) a consensus 16 rDNA sequence with a sequence identity of at least 99.5% to the polynucleotide sequence of SEQ ID NO: 21.

29. The preparation of claim 14, wherein the probiotic strain is Lactobacillus mucosae (Limosilactobacillus mucosae) DSM 33450 and exhibits the following characteristics:
a) a groL sequence with a sequence identity of 100% to the polynucleotide sequence of SEQ ID NO: 16;
b) a gyrB sequence with a sequence identity of 100% to the polynucleotide sequence according to SEQ ID NO: 17;
c) a dnaA sequence with a sequence identity of 100% to the polynucleotide sequence of SEQ ID NO: 18;
d) a rpsK sequence with a sequence identity of 100% to the polynucleotide sequence of SEQ ID NO: 19;
e) a rpmB sequence with a sequence identity of 100% to the polynucleotide sequence of SEQ ID NO: 20;
f) a consensus 16 rDNA sequence with a sequence identity of 100%, to the polynucleotide sequence according to SEQ ID NO: 21.

30. The preparation of claim 14, wherein the probiotic strain is Lactobacillus johnsonii DSM 33448 and exhibits the following characteristics:
a) a rpsK sequence with a sequence identity of at least 99.5% to the polynucleotide sequence of SEQ ID NO: 22;
b) a rpsM sequence with a sequence identity of at least 99.5% to the polynucleotide sequence of SEQ ID NO: 23;
c) a rplB sequence with a sequence identity of at least 99.5% to the polynucleotide sequence of SEQ ID NO: 24;
d) a sdhA sequence with a sequence identity of at least 99.5% to the polynucleotide sequence of SEQ ID NO: 25;
e) a rpmB sequence with a sequence identity of at least 99.5% to the polynucleotide sequence of SEQ ID NO: 26;
f) a consensus 16s rDNA sequence with a sequence identity of at least 99.5% to the polynucleotide sequence of SEQ ID NO: 27.

31. The preparation of claim 14, wherein the probiotic strain is Lactobacillus johnsonii DSM 33448 and exhibits the following characteristics:
a) a rpsK sequence with a sequence identity of 100%, to the polynucleotide sequence of SEQ ID NO: 22;
b) a rpsM sequence with a sequence identity of 100%, to the polynucleotide sequence of SEQ ID NO: 23;
c) a rplB sequence with a sequence identity of 100% to the polynucleotide sequence of SEQ ID NO: 24;
d) a sdhA sequence with a sequence identity of 100% to the polynucleotide sequence of SEQ ID NO: 25;
e) a rpmB sequence with a sequence identity of 100% to the polynucleotide sequence of SEQ ID NO: 26;
f) a consensus 16s rDNA sequence with a sequence identity of 100% to the polynucleotide sequence of SEQ ID NO: 27.

32. A food supplement or foodstuff comprising the preparation of claim 14 and at least one further ingredient.

33. The food supplement or foodstuff of claim 32, wherein the further ingredient is selected from the group consisting of: proteins; carbohydrates; fats; further probiotics; prebiotics; enzymes; vitamins; immune modulators; milk replacers; minerals; coccidiostats; acid-based products; medicines; and combinations thereof.
